(19) United States
(12) Patent Application Publication
(10) Pub. No.: US 2013/0084572 A1
(43) Pub. Date: Apr. 4, 2013

(54) CALIBRATIONS AND CONTROLS FOR DROPLET-BASED ASSAYS

(71) Applicants: Benjamin J. Hindson, Livermore, CA (US); Billy W. Colston, JR., San Ramon, CA (US); Kevin D. Ness, San Mateo, CA (US); Donald A. Masquelier, Tracy, CA (US)

(72) Inventors: Benjamin J. Hindson, Livermore, CA (US); Billy W. Colston, JR., San Ramon, CA (US); Kevin D. Ness, San Mateo, CA (US); Donald A. Masquelier, Tracy, CA (US)

(73) Assignee: QUANTALIFE, INC., Pleasanton, CA (US)

(21) Appl. No.: 13/251,016

(22) Filed: Sep. 30, 2011

(51) Int. Cl. G01N 21/75; G01N 21/64; C12Q 1/68

(52) U.S. Cl. 435/6.12; 436/164

(57) ABSTRACT

System, including methods and apparatus, for performing droplet-based assays that are controlled and/or calibrated using signals detected from droplets.

### CROSS-REFERENCES TO RELATED MATERIALS

This application incorporates by reference in their entireties for all purposes the following materials: U.S. Pat. No. 7,041,481, issued May 9, 2006; U.S. patent application Ser. No. 12/586,626, filed Sep. 23, 2009; U.S. patent application Ser. No. 12/976,827, filed Dec. 22, 2010; U.S. patent application Ser. No. 13/245,575, filed Sep. 26, 2011; U.S. Provisional Patent Application Ser. No. 61/194,043, filed Sep. 23, 2008; U.S. Provisional Patent Application Ser. No. 61/206,975, filed Feb. 5, 2009; U.S. Provisional Patent Application Ser. No. 61/271,538, filed Jul. 21, 2009; U.S. Provisional Patent Application Ser. No. 61/275,731, filed Sep. 1, 2009; U.S. Provisional Patent Application Ser. No. 61/277,200, filed Sep. 21, 2009; U.S. Provisional Patent Application Ser. No. 61/277,203, filed Sep. 21, 2009; U.S. Provisional Patent Application Ser. No. 61/277,204, filed Sep. 21, 2009; U.S. Provisional Patent Application Ser. No. 61/277,216, filed Sep. 21, 2009; U.S. Provisional Patent Application Ser. No. 61/277,249, filed Sep. 21, 2009; U.S. Provisional Patent Application Ser. No. 61/277,270, filed Sep. 22, 2009; and Joseph R. Lakowicz, PRINCIPLES OF FLUORESCENCE SPECTROSCOPY (2^{nd} Ed. 1999).

### INTRODUCTION

Droplet-based tests for amplification generally need to be accurate. If inaccurate, these tests can generate erroneous results, that is, false negatives and false positives. Each type of erroneous result can have detrimental consequences. False negatives related to detection of a disease could mean that the disease is not treated early and is permitted to spread. In contrast, false positives could cause unnecessary alarm, potentially triggering an unnecessary response that may be costly and disruptive. To avoid problems associated with false negatives and false positives, inaccurate amplification tests must be repeated to improve their reliability, which increases cost and uses more sample and reagent, each of which may be precious.

FIG. 1 shows a graph 5610 illustrating an exemplary approach for using fluorescence to measure amplification of a nucleic acid target in droplets formed by partitioning a sample. The graph plots, with respect to time, fluorescence signals that may be detected from a flow stream containing the droplets. Each droplet may be detected as a transient change (e.g., a transient increase) in intensity of the fluorescence signal, such as a peak or spike 5612 (i.e., a wave) formed by the fluorescence signal.

To improve clarity, the illustrative data shown here and in other figures of the present disclosure, are presented in a simplified form: each peak has no width and projects from a constant background signal 5613 formed by detection of a continuous phase carrying the droplets. However, a signal peak may have any suitable shape based on, for example, the frequency of detecting signals (the sampling rate), the shape of each droplet, the size and geometry of a channel carrying the flow stream, the flow rate, and the like. Moreover, the signal peaks may have any suitable temporal distribution, for example, occurring at relatively constant intervals, as shown here, or at varying intervals. A droplet signal provided by and/or calculated from the peak (e.g., a signal corresponding to peak height or peak area, among others) may be used to determine whether amplification occurred in the corresponding droplet, and thus whether the droplet received at least one molecule of the nucleic acid target when the sample was partitioned.

Each droplet signal may be compared to a signal threshold 5614, also termed a cutoff. This comparison may provide a determination of whether each droplet signal represents a positive signal (target is present) or a negative signal (target is absent and/or not detected), for amplification in the droplet. For example, droplet signals greater than (and, optionally, equal to) the threshold may be considered as representing positive droplets. Conversely, droplet signals less than (and, optionally, equal to) the threshold may be considered as representing negative droplets. (A positive droplet signal above threshold 5614 is indicated at 5616, and a negative droplet signal below threshold 5614 is indicated at 5618 in FIG. 1.) Comparison to the threshold thus may transform each droplet signal to a digital value, such as a binary value (e.g., a “1” for a positive droplet and “0” for a negative droplet). In any event, the fraction of droplets that are positive can be determined. For a given droplet size, the fraction of positive droplets can be used as an input to an algorithm based on Poisson statistics to determine the number of copies (molecules) of the nucleic acid target present in the initial sample volume. In some embodiments, more than one threshold may be used to categorize results (e.g., negative, positive, or inconclusive).

FIG. 2 shows an exemplary histogram 5620 of ranges of droplet signal intensities that may be measured from the flow stream of FIG. 1. The relative frequency of occurrence of each range is indicated by bar height. The distribution of positive and negative signal intensities may be larger than the modest difference in signal intensity produced by amplification (a positive droplet) relative to no amplification (a negative droplet). Thus, the distributions of droplet signals from positive droplets and negative droplets may produce a problematic overlap between the amplification-positive and amplification-negative droplet signals, indicated at 5624. Accordingly, as shown in FIG. 1, some amplification-positive droplets may provide relatively weak droplet signals, such as false-negative signal 5626, that are less than threshold 5614, resulting in incorrect identification of these positive droplets as negative. Conversely, some amplification-negative droplets may provide relatively strong droplet signals, such as false-positive signal 5628, that are greater than threshold 5614, resulting in incorrect identification of these negative droplets as positive. Since either type of erroneous result may be costly and harmful, it is desirable to minimize their occurrence.

There are many factors that can lead to variation in the fluorescence signal from droplets tested for amplification. Examples of physical parameters that may affect the fluorescence signal may include droplet position when detected (e.g., relative to the “sensed volume” of the detector), droplet volume and shape, optical alignment of detection optics (including excitation source, filters, and detector), detector response, temperature, vibration, and flow rate, among others. Examples of reaction chemistry parameters that may affect the fluorescence signal include the number of target molecules and/or the amount of background nucleic acid present in each droplet, amplification efficiency, batch-to-batch variations in reagent concentrations, and volumetric variability in reagent and sample mixing, among others. Variations in these physical and chemical parameters can increase the overlap in the distribution of positive and negative droplet signals, which can complicate data interpretation and affect test performance (e.g., affect the limit of detection). The variations can occur within a run and/or between runs, within a test on a target and/or between tests on different targets, on the same instrument and/or different instruments, with the same operator and/or different operators, and so on.

Thus, there is a need for improved accuracy and reliability in droplet-based amplification tests. For example, it would be desirable to have droplet-based controls for these tests, optionally, droplet-based controls that can be incorporated into test droplets or incorporated into control droplets that can be intermixed with test droplets. Such integrated controls may have the benefit of reducing cost by processing control reactions in parallel with test reactions, which may speed the analysis. It also would be useful to have one or more controls that can be used to verify hardware, reagent, and/or software (e.g., algorithm) performance.

### SUMMARY

The present disclosure provides a system, including methods and apparatus, for performing droplet-based assays that are controlled and/or calibrated using signals detected from droplets.

### BRIEF DESCRIPTION OF THE DRAWINGS

FIG. 1 is an exemplary graph of fluorescence signals that may be measured with respect to time from a flow stream of droplets, with the graph exhibiting a series of peaks representing droplet signals, and with the graph indicating a signal threshold for assigning droplet signals as corresponding to amplification-positive and amplification-negative droplets, in accordance with aspects of the present disclosure.

FIG. 2 is an exemplary histogram of ranges of droplet signal intensities that may be measured from the flow stream of FIG. 1, with the relative frequency of occurrence of each range indicated by bar height, in accordance with aspects of the present disclosure.

FIG. 3 is a flowchart illustrating an exemplary method of performing a droplet-based assay, in accordance with aspects of the present disclosure.

FIG. 4 is a flowchart illustrating an exemplary method of determining a concentration of a target, which may be performed as a step in the method of FIG. 3, in accordance with aspects of the present disclosure.

FIG. 5 is a schematic view of an exemplary system for performing droplet-based tests of nucleic acid amplification with the aid of controls and/or calibrators, in accordance with aspects of the present disclosure.

FIG. 6 is a schematic view of selected aspects of the system of FIG. 5, with the system in an exemplary configuration for detecting amplification of a nucleic acid target using a first dye, and for controlling for system variation during a test using a second dye, in accordance with aspects of present disclosure.

FIG. 7 is a schematic view of exemplary reagents that may be included in the system configuration of FIG. 6, to permit detection of amplification signals in a first detection channel and detection of a passive control signals in a second detection channel, in accordance with aspects of present disclosure.

FIG. 8 a flowchart of an exemplary approach to correcting for system variation using the system configuration of FIG. 6, in accordance with aspects of the present disclosure.

FIG. 9 is a schematic view of selected aspects of the system of FIG. 5, with the system in an exemplary configuration for detecting amplification of a nucleic acid target using a first dye in a set of droplets, and for (a) calibrating the system before, during, and/or after a test or (b) controlling for aspects of system variation during a test using either the first dye or a second dye in another set of droplets, in accordance with aspects of present disclosure.

FIG. 10 is an exemplary graph of fluorescence signals that may be detected over time from a flow stream of the system configuration of FIG. 9 during system calibration and sample testing performed serially, in accordance with aspects of present disclosure.

FIG. 11 is a flowchart of an exemplary method of correcting for system variation produced during a test using the system configuration of FIG. 9, in accordance with aspects of the present disclosure.

FIG. 12 is a schematic view of selected aspects of the system of FIG. 5, with the system in an exemplary configuration for testing amplification of a pair of nucleic acid targets in the same droplets, in accordance with aspects of present disclosure.

FIG. 13 is a schematic view of selected aspects of the system of FIG. 5, with the system in another exemplary configuration for testing amplification of a pair of nucleic acid targets in the same droplets, in accordance with aspects of present disclosure.

FIG. 14 is a schematic view of exemplary target-specific reagents that may be included in the system configurations of FIGS. 12 and 13, to permit detection of amplification signals in a different detection channel (i.e., a different detected wavelength or wavelength range) for each nucleic acid target, in accordance with aspects of present disclosure.

FIG. 15 is a pair of exemplary graphs of fluorescence signals that may be detected over time from a flow stream of the system configuration of FIG. 12 or 13 using different detection channels, with one of the channels detecting successful amplification of a control target, thereby indicating no inhibition of amplification, in accordance with aspects of present disclosure.

FIG. 16 is a pair of exemplary graphs with fluorescence signals detected generally as in FIG. 15, but with control signals indicating that amplification is inhibited, in accordance with aspects of present disclosure.

FIG. 17 is a schematic view of selected aspects of the system of FIG. 5, with the system in an exemplary configuration for testing amplification of a pair of nucleic acid targets using a different set of droplets for each target, in accordance with aspects of present disclosure.

FIG. 18 is a pair of exemplary graphs of fluorescence signals that may be detected over time from a flow stream of the system configuration of FIG. 17 using different detection channels, with each channel monitoring amplification of a distinct nucleic acid target, in accordance with aspects of present disclosure.

FIG. 19 is a pair of graphs illustrating exemplary absorption and emission spectra of fluorescent dyes that may be suitable for use in the system of FIG. 5, in accordance with aspects of the present disclosure.

FIG. 20 is a schematic diagram illustrating exemplary use of the fluorescent dyes of FIG. 19 in an exemplary embodiment of the system of FIG. 5, in accordance with aspects of the present disclosure.

FIG. 21 is a flowchart of an exemplary approach to correcting for system variation within a test by processing a set of droplet test signals to a more uniform signal intensity, in accordance with aspects of the present disclosure.

FIG. 22 is a flowchart of an exemplary approach for transforming droplet signals based on the width of respective signal peaks providing the droplet signals, in accordance with aspects of the present disclosure.

FIG. 23 is an exemplary graph of a signal that may be measured with respect to time from a fluid stream containing droplets, with individual peaks of the signal identified as positive droplets, negative droplets, or rejected droplets according to peak width and peak height, in accordance with aspects of the present disclosure.

FIG. 24 is a pair of graphs of exemplary first and second signals that may be measured with respect to time from a fluid stream containing droplets, with individual peaks of the second signal identified as positive droplets, negative droplets, or rejected droplets according to peak width of the first signal and peak height of the second signal, in accordance with aspects of the present disclosure.

FIG. 25 is a graph of an exemplary signal that may be measured with respect to time from a fluid stream containing droplets, with positive droplets forming signal peaks that are identified reliably and negative droplets forming signal peaks that cannot be identified reliably, in accordance with aspects of the present disclosure.

FIG. 26 is a pair of graphs of exemplary first and second signals that may be measured with respect to time from a fluid stream containing droplets, with positive droplets identified using the first signal and the total number of droplets identified using the second signal, in accordance with aspects of the present disclosure.

FIG. 27 is a series of graphs illustrating an exemplary assay-reporter signal and an exemplary droplet-marker signal, which collectively produce a combined signal that may be measured with respect to time in the same detection channel from a fluid stream containing droplets, in accordance with aspects of the present disclosure.

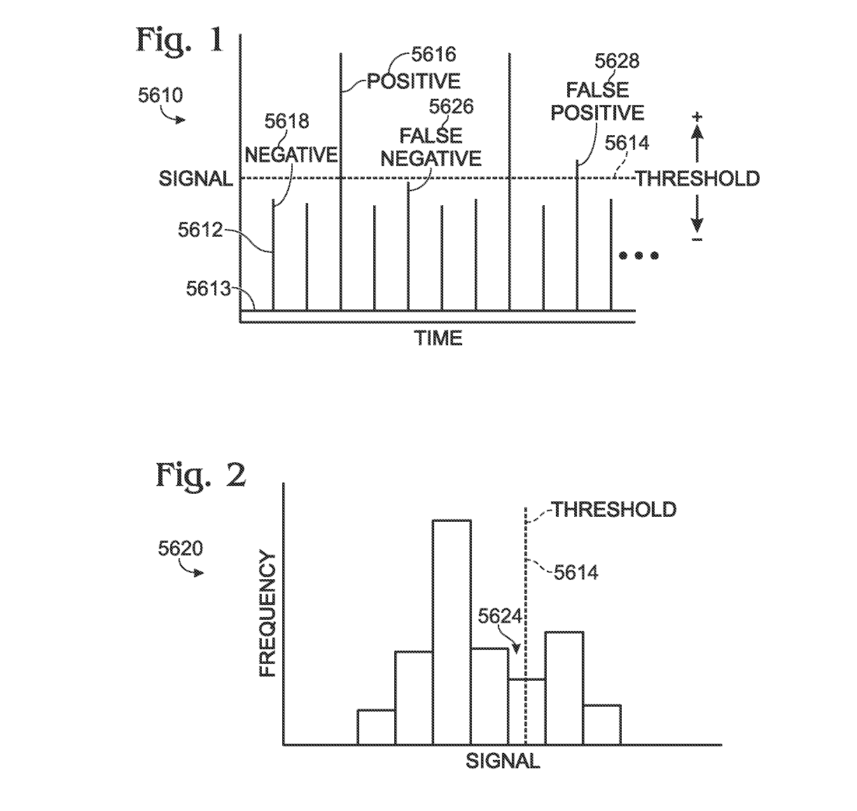

### DETAILED DESCRIPTION

The present disclosure provides a system, including methods and apparatus, for performing droplet-based assays that are controlled and/or calibrated using signals detected from droplets.

A method of performing a droplet-based assay is provided. In the method, a first signal and a second signal may be detected from a plurality of droplets. Accepted droplets of the plurality may be identified, with the first signal from the accepted droplets meeting a predefined condition. Rejected droplets of the plurality also may be identified, with the first signal from the rejected droplets failing to meet the predefined condition. A concentration of a target in the accepted droplets may be determined based on the second signal from the accepted droplets, and without any contribution of the second signal from the rejected droplets.

Another method of performing a droplet-based assay is provided. In the method, a plurality of droplets containing an assay reporter and a droplet marker may be generated. A signal may be detected from the plurality of droplets, with the signal representing combined emission of light from the assay reporter and the droplet marker. The assay reporter may provide a first integral portion of the signal having an intensity that varies according to whether or not a target is present in a droplet. The droplet marker may provide a second integral portion of the signal having an intensity that is at least substantially independent of whether or not the target is present in a droplet. A number of the plurality of droplets that are positive or a number that are negative for the target based on the signal may be counted. A total number for the plurality of droplets may be determined based on the signal. A concentration of the target may be obtained based on the counted number of droplets and the total number of droplets.

Yet another method of performing a droplet-based assay is provided. In the method, a signal may be detected from a plurality of droplets. Droplets positive for a target may be determined based on the signal. The positive droplets may be counted to establish a number of positive droplets. A total number for the plurality of droplets may be estimated. A concentration of the target may be obtained based on the number of positive droplets and the total number of droplets.

Still another method of performing a droplet-based assay is provided. In the method, a first signal may be detected from a plurality of droplets. Droplets positive for a target may be determined based on the first signal. The positive droplets may be counted to establish a number of positive droplets. A second signal may be detected from the plurality of droplets. The second signal may have an intensity corresponding to a size of each droplet and substantially independent of whether or not the target is present in such droplet. A total number for the plurality of droplets may be determined based on the second signal. A concentration of the target in the plurality of droplets may be obtained based on the number of positive droplets and the total number of droplets.

The present disclosure provides a method of sample analysis.

Droplets may be obtained. The droplets may be generated on-line or at least a subset of the droplets may be pre-formed off-line. At least a subset or all of the droplets may include a partition of a sample to be tested and may be capable of amplification of at least one test nucleic acid target, if present, in the partition. In some embodiments, the droplets may be capable of amplification of a test nucleic acid target and a control nucleic acid target. The droplets collectively or each may include a dye, or at least a first dye and a second dye. In some embodiments, the droplets may be of at least two types, such as two or more types of test droplets, test droplets and calibration droplets, or test droplets and control droplets, among others. In some embodiments, the two or more types of droplets may be distinguishable based on distinct temporal positions of the droplets types in a flow stream, the presence of respective distinct dyes in the droplet types, distinguishable signal intensities of the same dye (or different dyes), or a combination thereof, among others.

Signals, such as fluorescence signals, may be detected from the droplets. The signals may include test signals, calibration signals, control signals, reference signals, or any combination thereof. In some embodiments, test signals and control signals may indicate respectively whether amplification of a test nucleic acid target and a control nucleic acid target occurred in individual droplets. In some embodiments, detection may include (a) exciting first and second dyes with a same wavelength of excitation light and (b) detecting emitted light from the first and second dyes at least substantially independently from one another in respective first and second detection channels.

The signals detected may be analyzed to determine a test result related to a presence (number, concentration, etc.), if any, of a test nucleic acid target in the sample. In some embodiments, analysis may include transforming test signals based on reference signals to reduce variation in the test signals. The test signals and the reference signals may be detected in respective distinct detection channels or in the same detection channel. In some embodiments, the reference signals may be provided by a second dye that is not coupled to an amplification reaction and thus serves as a passive reference. In some embodiments, the reference signals may be provided by control signals detected from a control amplification reaction. The control amplification reaction may measure amplification of an exogenous or endogenous template. In some embodiments, analysis may include (a) comparing test signals, or a transformed set of the test signals, to a signal threshold to assign individual droplets as positive or negative for a test nucleic acid target, and (b) estimating a number of molecules of the test nucleic acid target in the sample based on the comparison. In some embodiments, analysis may include (a) analyzing control signals to determine a control value corresponding to a number and/or fraction of the droplets that are amplification-positive for a control nucleic acid target, and (b) interpreting a test result, such as determining its validity, based on the control value.

The systems disclosed herein may offer improved instrument calibration and/or substantial improvements in the accuracy and/or reliability of droplet-based amplification tests. Exemplary capabilities offered by the present disclosure may include any combination of (1) correcting/minimizing variations in the fluorescence signal to increase the accuracy of droplet PCR results; (2) providing an internal indicator of whether nucleic acid amplification failed (e.g., PCR inhibition from interfering components in the sample, incorrect sample and reagent mixing, incorrect thermal cycling, incorrect droplet formation); (3) providing measurement of droplet volumes without having to add additional hardware components; (4) providing measurement of changes in the baseline fluorescence signal (i.e., baseline drift); (5) providing calibration of a droplet detector before and/or during a run; (6) monitoring the performance of quantitative droplet PCR measurements and data processing algorithms before and/or during a run; (7) verification of droplet integrity (e.g., absence of coalescence); (8) obtaining information on droplet generation and detection frequency (spatially and temporally) using an in-line detector; (9) measuring variations and comparing them to predefined tolerances; (10) processing of raw droplet PCR data to correct for variations and increase test accuracy and performance; (11) incorporating control assays preferably using a single excitation source; and/or (12) quantifying one or more genetic targets by amplifying more than one genetic target in a single droplet.

Further aspects of the present disclosure are presented in the following sections: (I) definitions, (II) system overview, (III) exemplary instrument controls and calibrators, (IV) exemplary amplification controls, (V), exemplary multi-channel detection, (VI) exemplary self-normalization of test signals, and (VII) examples.

### I. DEFINITIONS

Technical terms used in this disclosure have the meanings that are commonly recognized by those skilled in the art. However, the following terms may have additional meanings, as described below.

Emulsion—a composition comprising liquid droplets disposed in an immiscible liquid. The droplets are formed by at least one dispersed phase, and the immiscible liquid forms a continuous phase. The continuous phase can also or alternatively be termed a carrier and/or a carrier phase. The dispersed phase (or at least one of the dispersed phases of a multiple emulsion) is immiscible with the continuous phase, which means that the dispersed phase (i.e., the droplets) and the continuous phase (i.e., the immiscible liquid) do not mix to attain homogeneity. The droplets are isolated from one another by the continuous phase and enclosed/surrounded by the continuous phase.

The droplets of an emulsion may have any uniform or non-uniform distribution in the continuous phase. If non-uniform, the concentration of the droplets may vary to provide one or more regions of higher droplet density and one or more regions of lower droplet density in the continuous phase. For example, droplets may sink or float in the continuous phase.

An emulsion may be monodisperse, that is, composed of droplets of uniform size, or may be polydisperse, that is, composed of droplets of various sizes. If monodisperse, the droplets of the emulsion may vary in size by a standard deviation of the volume (or diameter) that is less than about 50%, 20%, 10%, 5%, 2%, or 1% of the average droplet volume (or diameter). Droplets generated from an orifice may be monodisperse or polydisperse.

An emulsion may have any suitable composition. The emulsion may be characterized by the predominant liquid compound or type of liquid compound in each phase. The predominant liquid compounds in the emulsion may be one or more aqueous phases and one or more nonaqueous phases. The nonaqueous phase may be referred to as an oil phase comprising at least one oil, which generally includes any liquid (or liquefiable) compound or mixture of liquid compounds that is immiscible with water. The oil may be synthetic or naturally occurring. The oil may or may not include carbon and/or silicon, and may or may not include hydrogen and/or fluorine. The oil may be lipophilic or lipophobic. In other words, the oil may be generally miscible or immiscible with organic solvents. Exemplary oils may include at least one silicone oil, mineral oil, fluorocarbon oil, vegetable oil, or a combination thereof, among others.

In exemplary embodiments, the oil is a fluorinated oil, such as a fluorocarbon oil, which may be a perfluorinated organic solvent. A fluorinated oil may be a base (primary) oil or an additive to a base oil, among others. Exemplary fluorinated oils that may be suitable are sold under the trade name FLUORINERT (3M), including, in particular, FLUORINERT Electronic Liquid FC-3283, FC-40, FC-43, and FC-70. Another example of an appropriate fluorinated oil is sold under the trade name NOVEC (3M), including NOVEC HFE 7500 Engineered Fluid.

Droplet—a small volume of a first liquid that is enclosed by an immiscible second liquid, such as a continuous phase of an emulsion (and/or by a larger droplet). The volume of a droplet, and/or the average volume of droplets in an emulsion, may, for example, be less than about one microliter (or between about one microliter and one nanoliter or between about one microliter and one picoliter), less than about one nanoliter (or between about one nanoliter and one picoliter), or less than about one picoliter (or between about one picoliter and one femtoliter), among others. A droplet (or droplets of an emulsion) may have a diameter (or an average diameter) of less than about 1000, 100, or 10 micrometers, about 1000 to 10 micrometers, or about 500 to 1 micrometers, among others. A droplet may be spherical or nonspherical. A droplet may be a simple droplet or a compound droplet.

Surfactant—a surface-active substance capable of reducing the surface tension of a liquid in which it is present. A surfactant, which also or alternatively may be described as a detergent and/or a wetting agent, may incorporate both a hydrophilic portion and a hydrophobic portion, which may collectively confer a dual hydrophilic-hydrophobic character on the surfactant. A surfactant may, in some cases, be characterized according to its hydrophilicity relative to its hydrophobicity. Each dispersed and/or continuous phase may incorporate at least one surfactant. Each aqueous phase may include at least one nonionic surfactant and/or ionic surfactant. In some embodiments, the aqueous phase may include a surfactant that is a block copolymer of polypropylene oxide and polyethylene oxide. More particularly, the surfactant may be a block copolymer of polypropylene oxide and polyethylene oxide sold under the trade names PLURONIC and TETRONIC (BASF). In some embodiments, the surfactant may be a nonionic block copolymer of polypropylene oxide and polyethylene oxide sold under the trade name PLURONIC F-68. In some embodiments, the surfactant of the aqueous phase may be a water-soluble and/or hydrophilic fluorosurfactant. Exemplary fluorosurfactants for the aqueous phase are sold under the trade name ZONYL (DuPont), such as ZONYL FSN fluorosurfactants. In some cases, the surfactant may include polysorbate 20 (sold under the trade name TWEEN-20 by ICI Americas, Inc.). An exemplary concentration of surfactant for the aqueous phase is about 0.01 to 10%, 0.05 to 5%, 0.1 to 1%, or 0.5% by weight, among others.

A nonaqueous or oil phase may incorporate a hydrophobic surfactant. The nonaqueous phase may include one or more surfactants. The surfactants may include a nonionic surfactant, an ionic surfactant (a cationic (positively-charged) or anionic (negatively-charged) surfactant), or both types of surfactant. Exemplary anionic surfactants that may be suitable include carboxylates, sulphonates, phosphonates, and so on. The one or more surfactants may be present, individually or collectively, at any suitable concentration, such as greater than about 0.001% or 0.01%, or about 0.001% to 10%, 0.05% to 2%, or 0.05% to 0.5%, among others.

The one or more surfactants present in the nonaqueous phase (or oil phase) may be fluorinated surfactants (e.g., surfactant compounds that are polyfluorinated and/or perfluorinated). Exemplary fluorinated surfactants are fluorinated polyethers, such as carboxylic acid-terminated perfluoropolyethers, carboxylate salts of perfluoropolyethers, and/or amide or ester derivatives of carboxylic acid-terminated perfluoropolyethers. Exemplary but not exclusive perfluoropolyethers are commercially available under the trade name KRYTOX (DuPont), such as KRYTOX-FSH, the ammonium salt of KRYTOX-FSH (“KRYTOX-AS”), or a morpholino derivative of KRYTOX-FSH (“KRYTOX-M”), among others. Other fluorinated polyethers that may be suitable include at least one polyethylene glycol (PEG) moiety.

Fluorinated—including fluorine, typically substituted for hydrogen. Any of the fluorinated compounds disclosed herein may be polyfluorinated, meaning that such compounds each include many fluorines, such as more than five or ten fluorines, among others. Any of the fluorinated compounds disclosed herein also or alternatively may be perfluorinated, meaning that most or all hydrogens have been replaced with fluorine.

Analyte—a component(s) or potential component(s) of a sample that is analyzed in a test. An analyte is a specific subject of interest in a test where the sample is the general subject of interest. An analyte may, for example, be a nucleic acid, protein, peptide, enzyme, cell, bacteria, spore, virus, organelle, macromolecular assembly, drug candidate, lipid, carbohydrate, metabolite, or any combination thereof, among others. The analyte itself may be described as a target, or a target may represent the analyte. An analyte (and/or target) may be tested for any suitable aspect, such as its presence, activity, interaction with (e.g., binding to) one or more other components, and/or other characteristic in a sample and/or in partitions thereof. The presence of an analyte (and/or target) may relate to an absolute or relative number, concentration, binary assessment (e.g., present or absent), or the like, of the analyte (or target) in a sample or in one or more partitions thereof. In some examples, a sample may be partitioned (e.g., to create droplets) such that a copy of the analyte (or target) is not present in all of the partitions, such as being present in the partitions at an average concentration of about 0.0001 to 10,000, 0.001 to 1000, 0.01 to 100, or 0.1 to 10 copies (or molecules) per partition, or at an average concentration of less than about 10 copies, 2 copies, or 1 copy per partition. In some examples, a sample may be partitioned such that at least one or a plurality of the partitions include no copies (or molecules) of the analyte (or target) and/or such that at least one or a plurality of the partitions include only one copy (or molecule) of the analyte (or target).

Reaction—a chemical reaction, a binding interaction, a phenotypic change, or a combination thereof, which generally provides a detectable signal (e.g., a fluorescence signal) indicating occurrence and/or an extent of occurrence of the reaction. An exemplary reaction is an enzyme reaction that involves an enzyme-catalyzed conversion of a substrate to a product.

Any suitable enzyme reactions may be performed in the droplet-based assays disclosed herein. For example, the reactions may be catalyzed by a kinase, nuclease, nucleotide cyclase, nucleotide ligase, nucleotide phosphodiesterase, polymerase (DNA or RNA), prenyl transferase, pyrophospatase, reporter enzyme (e.g., alkaline phosphatase, beta-galactosidase, chloramphenicol acetyl transferse, glucuronidase, horse radish peroxidase, luciferase, etc.), reverse transcriptase, topoisomerase, etc.

Sample—a compound, composition, and/or mixture of interest, from any suitable source(s). A sample is the general subject of interest for a test that analyzes an aspect of the sample, such as an aspect related to at least one analyte that may be present in the sample. Samples may be analyzed in their natural state, as collected, and/or in an altered state, for example, following storage, preservation, extraction, lysis, dilution, concentration, purification, filtration, mixing with one or more reagents, pre-amplification (e.g., to achieve target enrichment by performing limited cycles (e.g., <15) of PCR on sample prior to PCR), removal of amplicon (e.g., treatment with uracil-d-glycosylase (UDG) prior to PCR to eliminate any carry-over contamination by a previously generated amplicon (i.e., the amplicon is digestable with UDG because it is generated with dUTP instead of dTTP)), partitioning, or any combination thereof, among others. Clinical samples may include and/or may be provided by a nasopharyngeal wash, blood, plasma, cell-free plasma, buffy coat, saliva, urine, stool, sputum, mucous, a wound swab, a tissue biopsy, milk, a fluid aspirate, a swab (e.g., a nasopharyngeal swab), and/or tissue, among others. Environmental samples may include water, soil, aerosol, and/or air, among others. Research samples may include cultured cells, primary cells, bacteria, spores, viruses, small organisms, any of the clinical samples listed above, or the like. Additional samples may include foodstuffs, weapons components, biodefense samples to be tested for bio-threat agents, suspected contaminants, and so on.

Samples may be collected for diagnostic purposes (e.g., the quantitative measurement of a clinical analyte such as an infectious agent) or for monitoring purposes (e.g., to determine that an environmental analyte of interest such as a bio-threat agent has exceeded a predetermined threshold).

Reagent—a compound, set of compounds, and/or composition that is combined with a sample in order to perform a particular test(s) on the sample. A reagent may be a target-specific reagent, which is any reagent composition that confers specificity for detection of a particular target(s) or analyte(s) in a test. A reagent optionally may include a chemical reactant and/or a binding partner for the test. A reagent may, for example, include at least one nucleic acid, protein (e.g., an enzyme), cell, virus, organelle, macromolecular assembly, drug candidate, lipid, carbohydrate, inorganic substance, or any combination thereof, and may be an aqueous composition, among others. In exemplary embodiments, the reagent may be an amplification reagent, which may include at least one primer or at least one pair of primers for amplification of a nucleic acid target, at least one probe and/or dye to enable detection of amplification, a polymerase, a ligase, nucleotides (dNTPs and/or NTPs), divalent magnesium ions, or any combination thereof, among others. In some embodiments, the reagent may be a PCR reagent, namely, a reagent involved in PCR amplification, such as a primer, a heat-stable polymerase, at least one nucleotide (dNTP or NTP), or magnesium, among others. An amplification reagent and/or a nucleic acid target each may be described as a reaction component.

The amplification reagent may be present at an effective amount, namely, an amount sufficient to enable amplification of a nucleic acid target in the presence of other necessary reagents. Exemplary effective amounts of PCR reagents are as follows: heat-stable DNA polymerase, 0.005 to 0.5 Units/μL; dNTPs, 50 μM to 5 mM each; primers, 0.02 to 5.0 μM each; and Mg²⁺, 0.5 to 10 mM.

Nucleic acid—a compound comprising a chain of nucleotide monomers. A nucleic acid may be single-stranded or double-stranded, among others. The chain of a nucleic acid may be composed of any suitable number of monomers, such as at least about ten or one-hundred, among others. Generally, the length of a nucleic acid chain corresponds to its source, with synthetic nucleic acids (e.g., primers and probes) typically being shorter, and biologically/enzymatically generated nucleic acids (e.g., nucleic acid analytes) typically being longer.

A nucleic acid may have a natural or artificial structure, or a combination thereof. Nucleic acids with a natural structure, namely, deoxyribonucleic acid (DNA) and ribonucleic acid (RNA), generally have a backbone of alternating pentose sugar groups and phosphate groups. Each pentose group is linked to a nucleobase (e.g., a purine (such as adenine (A) or guanine (T)) or a pyrimidine (such as cytosine (C), thymine (T), or uracil (U))). Nucleic acids with an artificial structure are analogs of natural nucleic acids and may, for example, be created by changes to the pentose and/or phosphate groups of the natural backbone. Exemplary artificial nucleic acids include glycol nucleic acids (GNA), peptide nucleic acids (PNA), locked nucleic acids (LNA), threose nucleic acids (TNA), and the like.

The sequence of a nucleic acid is defined by the order in which nucleobases are arranged along the backbone. This sequence generally determines the ability of the nucleic acid to bind specifically to a partner chain (or to form an intramolecular duplex) by hydrogen bonding. In particular, adenine pairs with thymine (or uracil) and guanine pairs with cytosine. A nucleic acid that can bind to another nucleic acid in an antiparallel fashion by forming a consecutive string of such base pairs with the other nucleic acid is termed “complementary.”

Replication—a process forming a copy (i.e., a direct copy and/or a complementary copy) of a nucleic acid or a segment thereof. Replication generally involves an enzyme, such as a polymerase and/or a ligase, among others. The nucleic acid and/or segment replicated is a template (and/or a target) for replication.

Amplification—a reaction in which replication occurs repeatedly over time to form multiple copies of at least one segment of a template molecule. Amplification may generate an exponential or linear increase in the number of copies as amplification proceeds. Typical amplifications produce a greater than 1,000-fold increase in copy number. Exemplary amplification reactions for the assays disclosed herein may include the polymerase chain reaction (PCR) or ligase chain reaction (LCR), each of which is driven by thermal cycling. Thermal cycling generally involves cycles of heating and cooling a reaction mixture to perform successive rounds of denaturation (melting), annealing, and extension. The assays also or alternatively may use other amplification reactions, which may be performed isothermally, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and the like. Amplification may utilize a linear or circular template.

Amplification may be performed with any suitable reagents. Amplification may be performed, or tested for its occurrence, in an amplification mixture, which is any composition capable of generating multiple copies of a nucleic acid target molecule (or region thereof), if present, in the composition. An amplification mixture may include any combination of at least one primer or primer pair, at least one probe, at least one replication enzyme (e.g., at least one polymerase, such as at least one DNA and/or RNA polymerase, and/or at least one ligase), and/or deoxynucleotide (and/or nucleotide) triphosphates (dNTPs and/or NTPs), among others.

PCR—nucleic acid amplification that relies on alternating cycles of heating and cooling (i.e., thermal cycling) to achieve successive rounds of replication. PCR may be performed by thermal cycling between two or more temperature set points, such as a higher melting (denaturation) temperature and a lower annealing/extension temperature, or among three or more temperature set points, such as a higher melting temperature, a lower annealing temperature, and an intermediate extension temperature, among others. PCR may be performed with a heat-stable polymerase, such as Taq DNA polymerase (e.g., wild-type enzyme, a Stoffel fragment, FastStart polymerase, etc.), Pfu DNA polymerase, S-Tbr polymerase, Tth polymerase, Vent polymerase, or a combination thereof, among others. PCR generally produces an exponential increase in the amount of a product amplicon over successive cycles.

Any suitable PCR methodology or combination of methodologies may be utilized in the assays disclosed herein, such as allele-specific PCR, assembly PCR, asymmetric PCR, digital PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, RT-PCR, single-cell PCR, solid-phase PCR, thermal asymmetric interlaced PCR, touchdown PCR, universal fast walking PCR, or any combination thereof, among others.

Amplicon—a product of an amplification reaction. An amplicon may be single-stranded or double-stranded, or a combination thereof. An amplicon corresponds to any suitable segment or the entire length of a nucleic acid target.

Primer—a nucleic acid capable of, and/or used for, priming replication of a nucleic acid template. Thus, a primer may be a shorter nucleic acid that is complementary to a longer template. During replication, the primer may be extended, based on the template sequence, to produce a longer nucleic acid that is a complementary copy of the template. Extension may occur by successive addition of individual nucleotides (e.g., by the action of a polymerase) or by attachment of a block of nucleotides (e.g., by the action of a ligase joining a pair of primers), among others. A primer may be DNA, RNA, an analog thereof (i.e., an artificial nucleic acid), or any combination thereof. A primer may have any suitable length, such as at least about 10, 15, 20, or 30 nucleotides. Exemplary primers are synthesized chemically. Primers may be supplied as at least one pair of primers for amplification of at least one nucleic acid target. A pair of primers may be a sense primer and an antisense primer that collectively define the opposing ends (and thus the length) of a resulting amplicon.

Probe—a nucleic acid connected to at least one label, such as at least one dye. A probe may be a sequence-specific binding partner for a nucleic acid target and/or amplicon. The probe may be designed to enable detection of target amplification based on fluorescence resonance energy transfer (FRET). An exemplary probe for the nucleic acid assays disclosed herein includes one or more nucleic acids connected to a pair of dyes that collectively exhibit fluorescence resonance energy transfer (FRET) when proximate one another. The pair of dyes may provide first and second emitters, or an emitter and a quencher, among others. Fluorescence emission from the pair of dyes changes when the dyes are separated from one another, such as by cleavage of the probe during primer extension (e.g., a 5′ nuclease assay, such as with a TAQMAN probe), or when the probe hybridizes to an amplicon (e.g., a molecular beacon probe). The nucleic acid portion of the probe may have any suitable structure or origin, for example, the portion may be a locked nucleic acid, a member of a universal probe library, or the like. In other cases, a probe and one of the primers of a primer pair may be combined in the same molecule (e.g., AMPLIFLUOR primers or SCORPION primers). As an example, the primer-probe molecule may include a primer sequence at its 3′ end and a molecular beacon-style probe at its 5′ end. With this arrangement, related primer-probe molecules labeled with different dyes can be used in a multiplexed assay with the same reverse primer to quantify target sequences differing by a single nucleotide (single nucleotide polymorphisms (SNPs)). Another exemplary probe for droplet-based nucleic acid assays is a Plexor primer. Some reagents that are termed “probes,” such as molecular inversion probes, may not include a label (e.g., may include no dye).

Label—an identifying and/or distinguishing marker or identifier connected to or incorporated into any entity, such as a compound, biological particle (e.g., a cell, bacteria, spore, virus, or organelle), or droplet. A label may, for example, be a dye that renders an entity optically detectable and/or optically distinguishable. Exemplary dyes used for labeling are fluorescent dyes (fluorophores) and fluorescence quenchers. The dye may be a compound, only part of a compound (i.e., a moiety), or the like.

The label may be a droplet marker that marks the position of each droplet, e.g., in a flow stream or field of view, among others. A droplet marker may have any suitable uniform or nonuniform distribution in each droplet. For example, the droplet marker may be distributed substantially uniformly throughout a droplet, may be localized to a perimeter of the droplet (e.g., localized to a skin that encapsulates the droplet), or may have one or more discrete localizations within the droplet (e.g., if the marker is a particle (such as a bead or quantum dot, among others)). Further aspects of a skin that encapsulates droplets are disclosed in U.S. patent application Ser. No. 12/976,827, filed Dec. 22, 2010, which is incorporated herein by reference.

Reporter—a compound or set of compounds that reports a condition, such as the extent of a reaction. Exemplary reporters comprise at least one dye, such as a fluorescent dye or an energy transfer pair, and/or at least one oligonucleotide. Exemplary reporters for nucleic acid amplification assays may include a probe and/or an intercalating dye (e.g., SYBR Green, ethidium bromide, etc.).

Binding partner—a member of a pair of members that bind to one another. Each member may be a compound or biological particle (e.g., a cell, bacteria, spore, virus, organelle, or the like), among others. Binding partners may bind specifically to one another. Specific binding may be characterized by a dissociation constant of less than about 10⁻⁴, 10⁻⁶, 10⁻⁸, or 10⁻¹⁰ M, among others. Exemplary specific binding partners include biotin and avidin/streptavidin, a sense nucleic acid and a complementary antisense nucleic acid (e.g., a probe and an amplicon), a primer and its target, an antibody and a corresponding antigen, a receptor and its ligand, and the like.

Signal—detectable and/or detected energy and/or information. Any of the signals detected, after detection, may be described as signals and/or data. For example, detected droplet signals may provide test signals and test data, control signals or control data, reference signals and reference data, calibration signals and calibration data, transformed signals and transformed data, or any combination thereof, among others. A signal may be detected optically, electrically, magnetically, mechanically, or the like.

Transform—to change one or more values, and/or the number, of signals of a data set using one or more mathematical and/or logical operations. Transformation of a set of signals may produce a transformed set of the signals by changing values of one or more of the signals and/or by deleting/invalidating any suitable subset of the signals. Signal transformation may include reducing signal variation, deleting/invalidating outlier signals, subtracting a baseline value from signals, reducing the frequency of outliers, reducing the overlap of distributions of positive and negative droplet signals, modifying signals according to a regression line, assigning new values to signals based on comparing signal values to a threshold or range, or any combination thereof, among others.

Run—an operating period during which a set of droplets, generally droplets of about the same size and including partitions a sample, are tested.

Oligonucleotide—a nucleic acid of less than about one-hundred nucleotides.

Exogenous—originating externally. For example, a nucleic acid exogenous to a sample is external to the sample as originally isolated. As another example, a nucleic acid exogenous to an organism or cell is not native to the organism or cell, such as a nucleic acid introduced into the organism or cell by infection or transfection.

Endogenous—originating internally, such as present in a sample as originally isolated or native to a cell or organism.

### II. SYSTEM OVERVIEW

FIG. 3 shows a flowchart illustrating an exemplary method 5640 of performing a droplet-based assay. The steps shown for the method may be performed in any suitable order and in any suitable combination, including combination with any other steps or features presented elsewhere in the present disclosure.

A sample for the assay may be prepared, indicated at 5642. The sample may be an aqueous sample and may contain at least one analyte to be tested in the assay. Preparation of the sample may include combining the sample, and particularly the analyte thereof, with one or more reagents, to create a reaction mixture. The reaction mixture may, for example, be an amplification mixture, such as a PCR composition.

Droplets may be generated, indicated at 5644. The sample and/or reaction mixture may be partitioned into the droplets. Accordingly, the sample and/or reaction mixture may be disposed in the droplets and/or contained by the droplets upon droplet generation. The sample may be partitioned to form a set of droplets with at least one or a plurality of the droplets having no copies of the analyte and/or at least one or a plurality of the droplets having only one copy of the analyte.

Generation of droplets may be performed by one or more droplet generators that each create droplets serially, or droplets may be produced in bulk, such as by agitation (e.g., sonication, blending, stirring, shaking, or the like). The droplets may be monodisperse or polydisperse. The droplets may be aqueous droplets disposed in a nonaqueous continuous phase (e.g., an oil phase). The droplets and/or the continuous phase may include a surfactant.

A reaction may be performed, indicated at 5646. The reaction may be performed in any of the droplets that are competent for the reaction. Stated differently, the droplets may be reacted, which means that droplets may be subjected to one or more conditions that promote occurrence of a reaction in the droplets. The reaction may, for example, occur preferentially or at least substantially exclusively in droplets that contain the analyte.

Any suitable reaction may be performed in droplets. The reaction may be a binding reaction, a chemical reaction, or a combination thereof, among others. The reaction may amplify at least one nucleic acid target, which may be the analyte(s) itself or a surrogate therefor. In some cases, the reaction may be performed by heating the droplets, such that they are incubated at an elevated temperature (above room temperature). For example, the reaction may be performed by thermally cycling the droplets (i.e., heating and cooling the droplets, and/or an emulsion in which the droplets are disposed, multiple times to execute a plurality of heating and cooling cycles). Thermal cycling may promote nucleic acid amplification, such as by PCR or the ligase chain reaction, among others. Thermal cycling may be achieved by moving the droplets through distinct temperature zones, such as with the droplets disposed in a continuous phase flowing along a channel that traverses the temperature zones. Alternatively, thermal cycling may be achieved with the droplets disposed in an emulsion held by a container, with emulsion not flowing and with the temperature of the container (and the emulsion therein) varied over time by heating and cooling.

A signal may be detected, indicated at 5648. The signal may be detected from an emulsion including the droplets and a continuous phase. The signal may be detected from any suitable number of droplets according to a desired accuracy and confidence for an assay. For example, an assay may detect a signal from at least about 10², 10³, 10⁴, or 10⁵ droplets, among others. Signal detection may be performed while the droplets are moving, such as traveling through a detection region (e.g., past a detection window) of a detector. The signal thus may be detected serially from the droplets. In some cases, the droplets may be carried to the detection region in a continuous phase that is flowing with the droplets through the detection region. The signal may be detected continuously from fluid (continuous phase plus droplets) or may be detected intermittently. If detected intermittently, the signal from a droplet may be detected at any suitable sampling frequency. In some cases, the signal may be detected as one or more images of droplets, such as an array of droplets in an emulsion. Accordingly, the signal may be detected from a plurality of the droplets concurrently and/or while the plurality of droplets are at least generally motionless.

The detected signal may be detected optically by measuring light, also termed optical radiation (i.e., ultraviolet light, visible light, and/or infrared light). For example, the signal may be a fluorescence signal. If two or more different fluorescence signals are measured from each droplet, the signals may, for example, be detected at distinct wavelengths or wavebands. Alternatively, the fluorescence signals may be measured at the same wavelength/waveband after excitation with different wavelengths or wavebands of light (e.g., excitation at different times or at different positions), among others. Two or more fluorescence signals may be detected from respective distinct fluorophores.

An aspect of an analyte may be determined, indicated at 5650. The aspect may be determined based on the signal(s) detected. The aspect may, for example, be a concentration, an activity, a conformation, an association, a modification, etc. The aspect may be in relation to the droplets and/or the sample. If a concentration is determined, the concentration may, for example, be expressed as molecules/copies per droplet, molecules/copies/moles per unit volume of sample, molecules/copies/moles in the original sample, or the like.

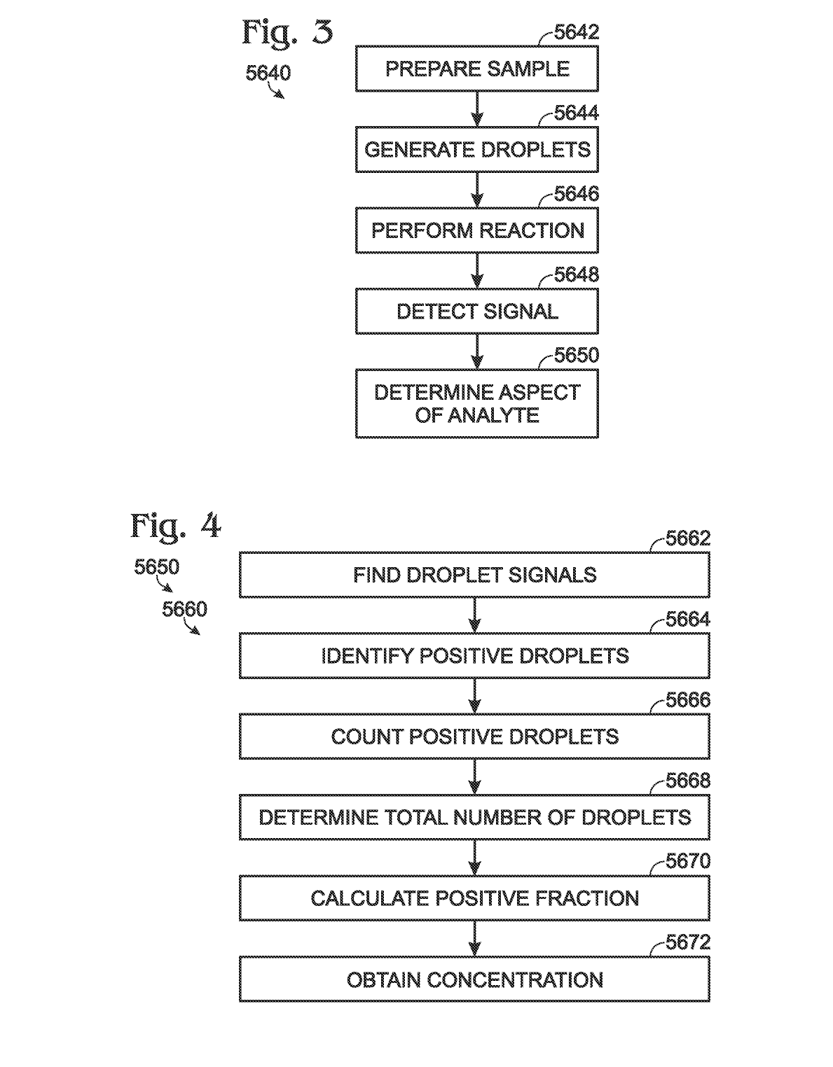

FIG. 4 shows a flowchart illustrating an exemplary method 5660 of determining a concentration of an analyte based on at least one detected signal, which may be performed as step 5650 of method 5640 of FIG. 3. The steps of method 5650 may be performed in any suitable order and in any suitable combination, including combination with any other steps or features presented elsewhere in the present disclosure.

Droplet signals may be found, indicated at 5662. In other words, a droplet- or peak-finding algorithm may be utilized to identify portions (e.g., peaks) of the signal detected from an emulsion or continuous phase and representing individual droplets within the emulsion or continuous phase. The droplet-finding algorithm may identify droplet signals according to any suitable criteria, such as comparing a signal from a peak with one or more predefined conditions for peak height, peak shape, peak width, or a combination thereof, among others. In some cases, each droplet signal may be identified by comparing the detected signal with one or more thresholds (e.g., using a signal characteristic described above for the positive/negative threshold). For example, the maximum or total intensity of the signal (e.g., the peak height or peak area) from a negative droplet may be compared with a threshold value, to distinguish the droplet from noise (e.g., see Example 2). The algorithm may be capable of identifying a droplet signal for each droplet (e.g., without distinguishing whether the droplet is positive or negative) or only for droplets that produce a stronger signal (e.g., positive droplets only or positive droplets and only a subset of negative droplets).

Positive droplets may be identified, indicated at 5664. Droplets that test positive for a reaction (and thus for the presence of the analyte or target) may be identified by comparing each droplet signal identified (from 5662) with a predefined condition, such as a threshold, that distinguishes positive droplets from negative droplets. For example, the maximum intensity (and/or the peak height) of the droplet signal detected from a droplet may be compared with a threshold (also termed a threshold value), to classify the droplet as positive or negative. In other examples, the signal for a droplet may be integrated, averaged, smoothed, and/or the like, and then compared with one or more predefined conditions (e.g., one or more thresholds) to distinguish positive droplets from negative droplets.

Positive droplets may be counted, indicated as 5666. More particularly, droplet signals identified as corresponding to positive droplets may be counted to determine how many droplets from the complete set analyzed test positive for a reaction and/or a target, among others. In some cases, counting positive droplets may find the number of droplets analyzed that contain an analyte/target molecule. In some cases, negative droplets (i.e., droplets that are non-positive and not excluded) may be counted instead of positive droplets, because either the number of positive droplets or the number of negative droplets may be used for subsequent steps.

The total number of droplets may be determined, indicated at 5668. The total number represents both the positive (or negative) droplets that were counted at 5666 and negative (or positive) droplets that were not counted. The total number of droplets may be determined by counting both negative droplets and positive droplets, if droplet signals for both types of droplets were identified efficiently at 5662. Alternatively, the total number of droplets may be estimated. Further aspects of determining the total number of droplets by counting peaks and by estimation are described below in Example 2.

A fraction of the total number of droplets that are positive, or a fraction that are negative, may be calculated, indicated at 5670. The fraction may be calculated as the number of counted positive (or negative) droplets (at 5666) divided by the total number of droplets determined (at 5668). Alternatively, a negative (or positive) fraction may be calculated as the number of counted negative (or positive) droplets divided by the total number of droplets determined, and then the positive (or negative) fraction can be calculated as one minus the negative (or positive) fraction.

The concentration of the analyte may be obtained, indicated at 5672. The concentration may be expressed with respect to the droplets and/or with respect to a sample disposed in the droplets and serving as the source of the analyte. The concentration of the analyte in the droplets may be calculated from the fraction of positive (or negative) droplets by assuming that analyte molecules have a Poisson distribution among all of the droplets. With this assumption, the fraction f(k) of droplets having k copies of the analyte is given by the following equation:

f(k)=(C^{k}/k!)exp(−C)

Here, C is the concentration of analyte in the droplets, expressed as the average number of analyte copies/molecules per droplet. Simplified Poisson equations may be derived from the more general equation above and used to determine analyte concentration from the fraction of positive (or negative) droplets. An exemplary Poisson equation that may be used is as follows:

C=−ln(1−fₚ)

where fₚ is the fraction of positive droplets analyte (i.e., fₚ=f(1)+f(2)+f(3)+ . . . ), which is a measured estimate of the probability of a droplet having at least one copy of the. Another exemplary Poisson equation that may be used is as follows:

C=−ln(fₙ)

where fₙ is the fraction of negative droplets (or 1−fₚ), which is a measured estimate of the probability of a droplet having no copies of the analyte, and C is the concentration as described above.

In some embodiments, an estimate of the concentration of the analyte may be obtained directly from the positive fraction, without use of a Poisson equation. In particular, the positive fraction and the concentration converge as the concentration decreases. For example, with a positive fraction of 0.1, the concentration is determined with the above equation to be about 0.105, a difference of only 5%; with a positive fraction of 0.01, the concentration is determined to be about 0.01005, a ten-fold smaller difference of only 0.5%. However, use of the Poisson equation can provide a more accurate estimate of concentration, particularly with a relatively higher positive fraction, because the equation accounts for the occurrence of multiple analyte copies/molecules per droplet.

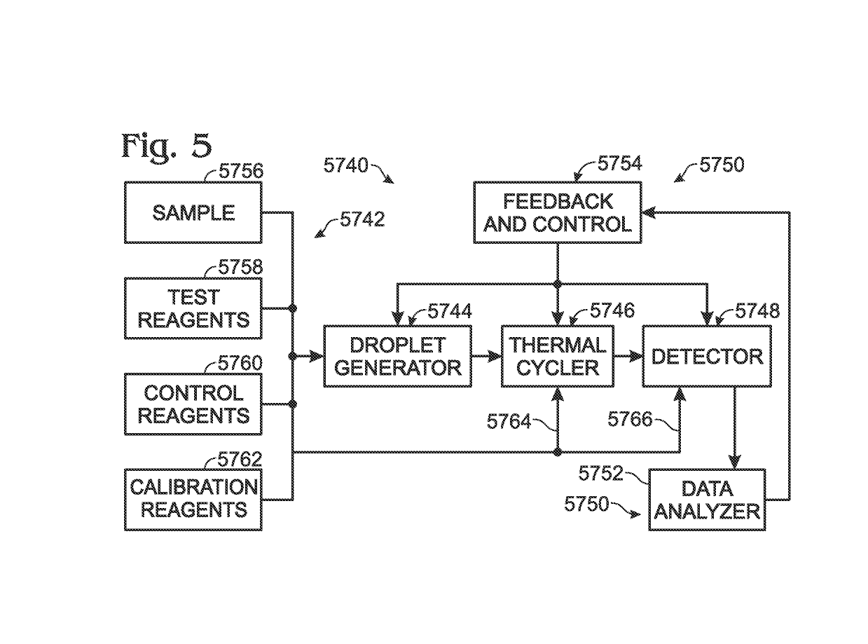

FIG. 5 shows an exemplary system 5740 for performing droplet-based tests of nucleic acid amplification with the aid of controls and/or calibrations. System 5740 may include any combination of a sample/reagent storage/preparation assembly 5742, at least one droplet generator 5744, an amplification assembly, such as a thermal cycler 5746, a detection assembly 5748, and a controller 5750 incorporating a data analyzer 5752 and a feedback and control portion 5754, among others.

The system may provide at least one flow stream that carries at least one sample and reagents from one or more upstream positions and in a downstream direction to detection assembly 5748. Signals detected from the flow stream, and particularly droplet signals, may be communicated to data analyzer 5752. The data analyzer may analyze the signals to determine one or more test results, control results, calibration results, a quality (e.g., validity, reliability, confidence interval, etc.) of any of the results, or a combination thereof. Any of the results may be communicated to feedback and control portion 5754, which may control and/or adjust control of any of storage/preparation assembly 5742, droplet generator 5744, thermal cycler 5746, detection assembly 5748, and data analyzer 5752, based on the results determined.

Storage/preparation assembly 5742 may contain and/or supply at least one sample 5756, at least one set of test reagents 5758 (also termed target reagents), one or more control reagents 5760, one or more calibration reagents 5762, or any combination thereof. Any of the samples and/or reagents may be stored and/or supplied separately, may be stored and/or supplied as one or more pre-formed mixtures, and/or may be mixed selectably before they are supplied to a downstream region of the system (e.g., droplet generator 5744, thermal cycler 5746, or detection assembly 5748). Furthermore, any of the samples and/or reagents may travel sequentially from storage/preparation assembly 5742 to droplet generator 5744, thermal cycler 5746, and then detection assembly 5748 for detection of droplet signals. Alternatively, any of the samples and/or reagents may reach the detection assembly without travel through the droplet generator, as indicated at 5764, the thermal cycler, or both, as indicated at 5766. Accordingly, any of the samples and/or reagents disclosed herein may be stored and/or supplied in pre-formed droplets. Droplets may, for example, be pre-formed off-line, either locally or remotely. Pre-formed droplets may be intermixed randomly with droplets formed by droplet generator 5744 before reaching detection assembly 5748, or distinct types of droplets may be detected as spatially and/or temporally separated sets of droplets.

Test reagents 5758 are any reagents used to test for amplification of one or more targets, such as one or more primary targets, in partitions of a sample. Primary targets generally comprise any targets that are of primary interest in a test. Primary targets may be present at an unknown level in a sample, prior to performing tests on the sample. Test reagents 5758 generally include one or more sets of target reagents conferring specificity for amplification of one or more particular nucleic acid targets to be tested in a sample. Thus, the test reagents may include at least one pair (or two or more pairs) of primers capable of priming amplification of at least one (or two or more) nucleic acid target(s). The test reagents also may comprise at least one reporter to facilitate detecting amplification of each test target, a polymerase (e.g., a heat stable polymerase), dNTPs, and/or the like. The test reagents enable detection of test signals from droplets.

Control reagents 5760 are any reagents used to control for test signal variation (generally, variation other than that produced by differences in amplification) and/or to interpret results obtained with the test reagents (such as a reliability and/or validity of the results). The control reagents permit control signals and/or reference signals to be detected from droplets, either the same or different droplets from the test signals. Control reagents may be mixed with test reagents prior to droplet formation and/or control droplets containing control reagents may be produced separately from the test droplets and introduced independently of the sample.

The control reagents may provide instrument controls, that is, controls for variation introduced by the system (and/or its environment). Thus, instrument controls may control for variation in droplet volume, droplet detection efficiency, detector drift, and the like. Reference signals may be detected from droplets containing control reagents that function as instrument controls.

The control reagents also or alternatively may provide amplification controls, that is, controls that test for secondary/control amplification in droplets. The control reagents thus may include reagents used to test for amplification of at least one secondary or control target in droplets. The secondary/control target may be of secondary interest in a test, and/or may be present at a known or expected level in the sample, among others. In any event, the control reagents may include one or more sets of target reagents conferring specificity for amplification of one or more control nucleic acid targets to be tested in droplets. The control reagents may include at least one pair (or two or more pairs) of primers capable of priming amplification of at least one (or two or more) control nucleic acid target(s). The control reagents also may comprise at least one reporter to facilitate detecting amplification of each control target, a polymerase (e.g., a heat stable polymerase), dNTPs, and/or the like, or any suitable combination of these control reagents may be supplied by the test reagents. Control signals may be detected from control reagents that function as amplification controls.

Calibration reagents 5762 are any reagents used to calibrate system operation and response. Droplets containing a calibration reagent (i.e., calibration droplets) may be introduced into a flow stream of the system, at any position upstream of the detection assembly, for the purpose of calibrating the system (e.g., calibrating flow rates, excitation power, optical alignment, detector voltage, amplifier gain, droplet size, droplet spacing, etc.). Calibration droplets may be introduced into a flow stream of the system before, during, and/or after introduction of test droplets into the flow stream. In some embodiments, the level of a dye within control droplets may be used to calibrate and/or validate detector response, such as by using a pair of dye concentrations providing calibration signals that bracket an intended measuring range and/or that are disposed near upper and lower ends of the measuring range. For example, droplets of known size and containing one or more known dye concentrations may be prepared off-line and introduced into the system, and/or may be generated by the system. In some embodiments, calibration droplets may comprise fluorescent particles such as quantum dots, polymer beads, etc.

System 5740 may used to perform a method of analyzing one or more samples. The method may include any suitable combination of the steps disclosed herein, performed in any suitable order.

Droplets may be obtained. The droplets may be of one type or two or more types. At least a subset, or all, of the droplets may be generated by the system or may be pre-formed off-line. At least a subset of the droplets may include test reagents for testing amplification of a test nucleic acid target. At least a subset of the droplets may include control reagents and/or calibration reagents for testing amplification of a control nucleic acid target. The droplets may contain one or more dyes.

The droplets may be introduced into a flow stream upstream of a detector. All of the droplets may be introduced into the flow stream at the same position or the droplets, particularly droplets of different types, may be introduced at two or more distinct positions.

The droplets, in the flow stream, may be subjected to conditions that facilitate amplification. For example, the droplets may be heated and/or may be heated and cooled repeatedly (thermally cycled).

Signals may be detected from the droplets. The signals may include test signals, control signals, reference signals, calibration signals, or any combination thereof.

The signals may be analyzed. Analysis may include transforming test signals. Analysis also or alternatively may include comparing test signals and/or transformed test signals to a signal threshold to assign individual droplets as being positive or negative for amplification of a nucleic acid target. A number and/or fraction of target-positive droplets may be determined based on results of the comparison. Analysis further may include estimating a presence of a nucleic acid target in the sample. The estimated presence may be no target in the sample. Estimation of the presence may (or may not) be performed using Poisson statistics.

### III. EXEMPLARY INSTRUMENT CONTROLS AND CALIBRATORS

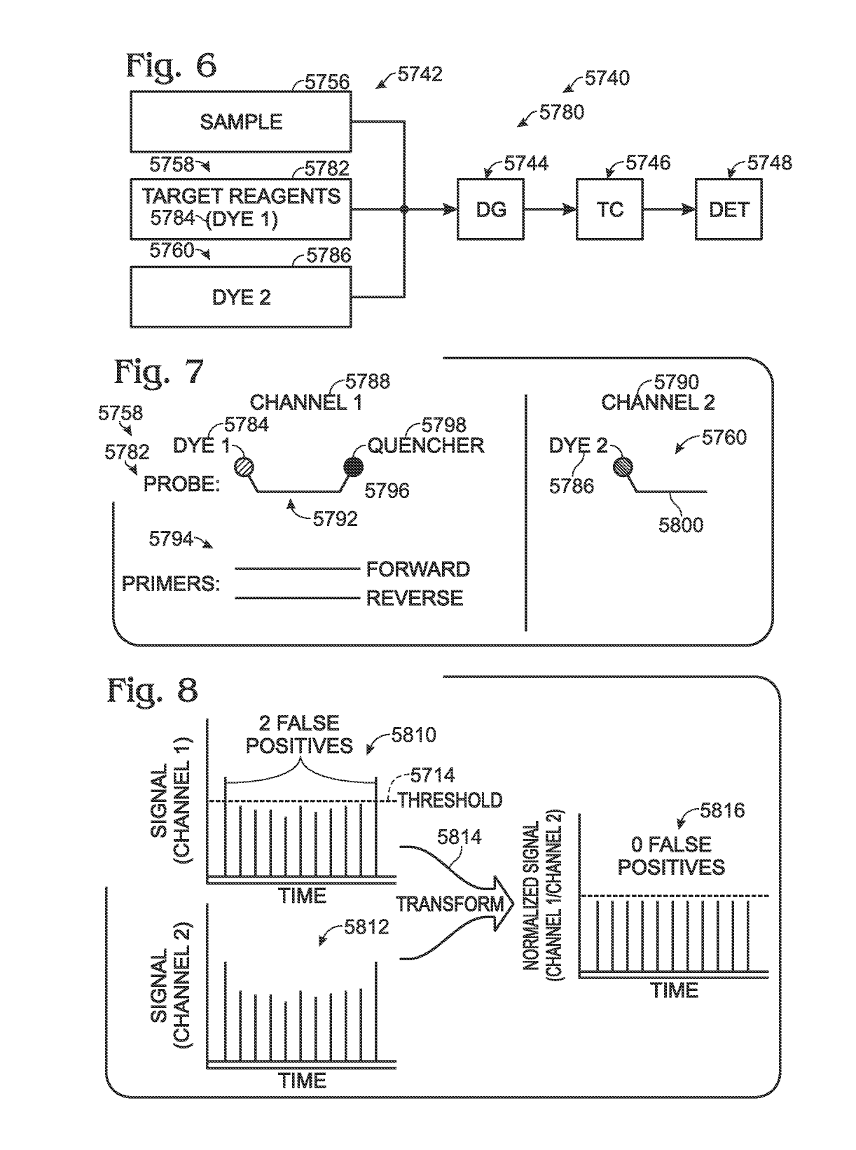

FIG. 6 shows selected aspects of system 5740 in an exemplary configuration 5780 for detecting amplification of a nucleic acid target using a first dye and for controlling for system variation during a test using a second dye. In FIG. 6 and in other system configurations presented in succeeding figures of the present disclosure, the terms “droplet generator,” “thermal cycler,” and “detection assembly” are abbreviated “DG,” “TC,” and “DET.”

Storage/preparation assembly 5742 may supply an amplification mixture to droplet generator 5744. The amplification mixture may incorporate a sample 5756, target reagents 5782 (i.e., test reagents 5758) including a first dye 5784 (dye 1), and a second dye 5786 (dye 2). The second dye and the target reagents may be mixed with one another before introduction into system 5740 or may be mixed within the system. Target reagents 5782 may provide primers for amplification of a nucleic acid target, and the first dye may enable detection of whether amplification occurred. The first and second dyes may be fluorescent dyes that are distinguishable optically. The second dye may be a passive reference or instrument control. In other words, the second dye may provide a detectable signal having an intensity that is at least substantially independent of the extent of amplification, if any, of any nucleic acid target. In some cases, the second dye may be a droplet marker for identification of droplet positions within a continuous phase.

Droplet generator 5744 may form droplets of the amplification mixture. The droplets may travel through thermal cycler 5746, to promote amplification of the nucleic acid target, if any, in each droplet. The droplets then may travel to detection assembly 5748. Assembly 5748 may detect, for each droplet, a test signal from the first dye and a reference signal (also termed a control signal) from the second dye.

FIG. 7 shows exemplary target reagents 5782 and a control reagent 5760 that may be included in system configuration 5780 of FIG. 6. The target and control reagents may permit detection of test signals in a first detection channel 5788 (“channel 1”) and detection of reference signals in a second detection channel 5790 (“channel 2”). The first and second channels may represent distinct wavelengths and/or at least substantially nonoverlapping wavelength ranges.

Target reagents may include a reporter, such as a probe 5792, and target-specific forward and reverse primers 5794. Probe 5792 may be an energy transfer probe (e.g., a TAQMAN probe) including a nucleic acid, such as an oligonucleotide 5796, that binds to amplified target, and an energy transfer pair connected to strand 5796. The energy transfer pair may, for example, be formed by first dye 5784 and a quencher 5798.

Control reagent 5760 may include second dye 5786. The second dye may (or may not) be connected to a nucleic acid, such as an oligonucleotide 5800. Connection to the oligonucleotide may be covalent and/or through a binding interaction. Connection of the second dye to an oligonucleotide or other water-soluble molecule may improve retention of the second dye in the aqueous phase of a droplet and/or may facilitate distribution of the dye throughout the aqueous phase, among others.

FIG. 8 shows a flowchart illustrating of an exemplary approach to correcting for system variation using system configuration 5780 (FIG. 6), and, optionally, the reagents illustrated in FIG. 7. Test signals (i.e., target signals) and reference signals may be detected from the same droplets. For example, test signals may be detected in a first channel and reference signals may be detected in a second channel. Graphs illustrating coincident detection of test signals and reference signals are shown at 5810, 5812, respectively.

Test signal variation may introduce errors in data processing. For example, graph 5810 shows substantial variation in the intensity of the test signals detected. As a result, some of the test signals may be erroneously classified as positives or negatives. In the present illustration, two false positives are marked. However, variation of the test signals may be mirrored by variation of the reference signals detected from the same droplets. Accordingly, the test signals may be transformed based on the reference signals, indicated at 5814, to correct for variation in the test signals, as shown in a graph 5816, which plots the transformed test signals. The test signals may be transformed by any suitable operation or set of operation involving the reference signals. For example, the test signals may be transformed through dividing test signals by reference signals, such as dividing each test signal by its corresponding reference signal, which may be described as normalizing the test signals. Alternatively, the test signals may be transformed based on the reference signals by, for example, baseline subtraction, distance from the regression line, or the like. A transformation may compensate for variations in the test channel. This compensation or correction may make the test signals (i.e., negative test signals and/or positive test signals) more uniform in value and/or more Gaussian. The transformation also or alternatively may reduce the frequency of outliers and/or the overlap of the distributions of positive and negative signals.

FIG. 9 shows selected aspects of system 5740 in an exemplary configuration 5830 for (a) detecting amplification of a nucleic acid target in a set of droplets and (b) system calibration and/or correction for system variation in another set of droplets. Configuration 5830 is similar to configuration 5780 of FIG. 6, except that target reagents 5782 and control reagent 5760 are not in the same droplets. Accordingly, the target reagents and the control reagent may be supplied to respective distinct droplet generators of the system, indicated at 5832, may be supplied to the sample droplet generator at different times, or the control reagent may be supplied in pre-formed droplets that do not pass through the droplet generator, indicated at 5834, 5836. Since the target reagents and the control reagent are not in the same droplets in this configuration, the control reagent may include the same dye as the target reagent (i.e., first dye 5784) or may include a distinct dye (such as second dye 5786).

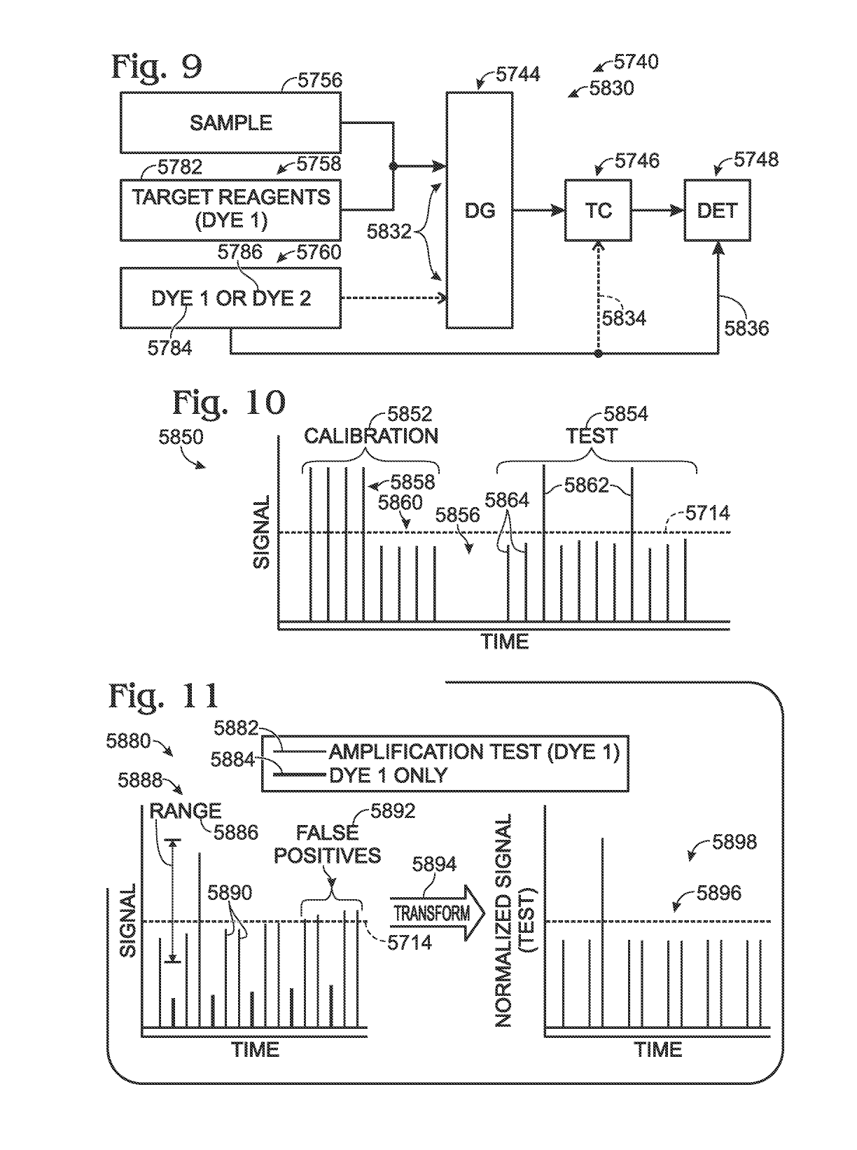

FIG. 10 shows an exemplary graph 5850 of fluorescence signals that may be detected over time from a flow stream of system configuration 5830 (FIG. 9) during system calibration, indicated at 5852, and sample testing, indicated at 5854. Calibration and sample testing may be performed without or with mixing of calibration and test droplets.

Calibration and sample testing may be performed serially, without mixing of droplet types, using the same dye (and/or detection of the same wavelength(s)). By keeping calibration and test droplets separate, the distributions of test and calibration signal intensities may overlap. For example, calibration droplets and test droplets may be separated temporally in the flow stream, such that each type of droplet is identifiable based on its time of arrival at the detection assembly. The time of arrival may be calculated based on the relative time of introduction of each droplet type into the flow stream and the velocity of the flow stream. Thus, the calibration and test droplets may not (or may) be distinguishable based on signal intensity, but may be distinguishable temporally. In particular, the test and calibration droplets may be separated by a temporal (and spatial) gap 5856, which may identify a transition between droplet types. The use of temporal gaps also may permit introduction of a set of calibration droplets within a set of test droplets (i.e., within a test run), with a gap preceding and following the set of calibration droplets, to provide identification of each transition to a different droplet type. Stated differently, calibration may be performed during sample testing, by inserting calibration droplets into a train of test droplets, such that the train of test droplets is divided into two or more discrete groups.

Calibration droplets may include two or more types of droplet, which may be introduced separately or intermixed. For example, FIG. 10 shows a set of stronger calibration signals 5858 followed by a set of weaker calibration signals 5860 produced by distinct types of calibration droplets. Stronger and weaker calibration signals 5858, 5860 may correspond generally in intensity to respective positive test signals 5862 and negative test signals 5864. In other embodiments, only one type or three or more types of calibration droplet may be used, and may be configured respectively to provide one or three or more intensities of calibration signals.

Calibration and sample testing alternatively may be performed with calibration and test droplets randomly intermixed and thus not distinguishable temporally. Intermixed calibration and test droplets may be distinguishable by incorporating distinguishable dyes into the respective droplet types and, optionally, by detection of the distinguishable dyes at respective distinct wavelengths. Alternatively, or in addition, calibration droplets and test droplets may be distinguishable according to signal intensity detected at the same wavelength(s) and optionally from the same dye. In particular, calibration droplets may be designed to have one or more signal intensities outside the signal range of test droplets (i.e., the signal range provided by the collective distribution of signal intensities from negative and positive test droplets (e.g., see FIG. 2)). Thus, calibration droplets may be identified based on their calibration signals having signal intensities above and/or below the signal range of test droplets.

FIG. 11 shows a flowchart 5880 of an exemplary approach to correcting for signal variation during an amplification test using system configuration 5830 of FIG. 9. The approach illustrated in FIG. 11 distinguishes types of droplet signals, namely, test droplet signals 5882 and reference droplet signals 5884, based on differences in signal intensity detected in the same detection channel, as described above for calibration droplets. In particular, test droplets may produce a range 5886 of signal intensities, and reference signals 5884 may have intensities below (or above) the range. Accordingly, the distinct types of droplets may be interspersed randomly in the flow stream.

The reference droplets may be formed with the same amount (or two or more discrete amounts) of dye. Accordingly, without signal variation generated by the system, the reference droplets should produce reference signals of the same intensity. Variation in reference signal intensity may be mirrored by corresponding changes in the intensity of test signals. For example, in graph 5888, the intensity of reference signals 5884 and negative test signals 5890 show a gradual increase with respect to time. As a result, test signals from amplification-negative droplets may produce false positives 5892.

Variation in test signals 5882 may be reduced by transforming the test signals, indicated at 5894, based on reference signals 5884, to produce normalized test signals 5896 presented in graph 5898. Transformation may, for example, be performed by transforming each test signal based on one or more reference signals temporally proximate to the test signal, a weighted average of reference signals temporally proximate to the test signal, a sliding window of averaged reference signals that overlaps the test signal, or the like. Transformation before comparing test signals to a threshold may reduce the incidence of false positives, as shown here, the incidence of false negatives, or both.

### IV. EXEMPLARY AMPLIFICATION CONTROLS

FIG. 12 show selected aspects of system 5740 of FIG. 5, with the system in an exemplary configuration 5910 for testing amplification of at least a pair of nucleic acid targets in the same droplets. System configuration 5910 may form an amplification mixture, which is supplied to droplet generator 5744. The amplification mixture may incorporate a sample 5756, test amplification reagents 5858, control amplification reagents 5912, and at least one control template 5914. Any combination of the sample, test reagents, control reagents, and control template may be mixed with one other before introduction into system 5740, or may be mixed within the system. Test reagents 5758 and control reagents 5912 may provide primers for respective amplification of at least one test target and at least one control target.

Amplification of the test and control targets may, for example, be detected via a first dye and a second dye, respectively, which may be included in respective first and second reporters (e.g., first and second probes). Signals from the first and second dyes may be detected in distinct (e.g., at least substantially nonoverlapping) first and second channels (i.e., a test channel and a control channel) as test signals and control signals, respectively.

Control template 5914 may comprise exogenous molecules of the control target. In contrast, the sample may be tested for a presence of endogenous molecules of the test target. The control template 5914 may be present in any suitable amount to provide any suitable average number of control template molecules per droplet, to generate a desired fraction of droplets positive for the control template. For example, the number of template molecules provided by template 5914 may be substantially less than an average of one per droplet, such as an average of about 0.1, 0.05, 0.02, or 0.01 molecule per droplet. Accordingly, the number/concentration of control template molecules may be selected such that the frequency of amplification of both test and control targets in the same droplet is low, which may minimize competition that may be caused by amplification of both test and control targets. For example, the control template may be present in no more than about one in five droplets.

The frequency of amplification of the control target may be determined by performing an analysis with the system. In some embodiments, this frequency may be compared with one or more previously determined frequencies of amplification for the control target and/or may be compared with an expected value for the frequency provided by a manufacturer. In any event, a control value may be determined, with the control value corresponding to a number and/or fraction of the droplets that are amplification-positive for the control nucleic acid target.

Control signals acquired in the control channel may be used to measure and/or verify the quantitative accuracy of a run and/or the measurement precision of the system during two or more runs. The control signals also or alternatively may be used to interpret a test result, such as the quality of test data measured from a sample, for example, to verify the quantitative accuracy of the test data and/or to determine the validity and/or reliability of the test data. The test result may be interpreted based on control value determined. For example, the test result may be determined as being invalid if the control value is less than a threshold value. Furthermore, data acquired from the control channel, such as signals from amplification-negative control droplets, may provide reference signals, as described above in relation to FIG. 8. In other words, test signals may be transformed using control signals that functions as reference signals, to normalize the test signals.

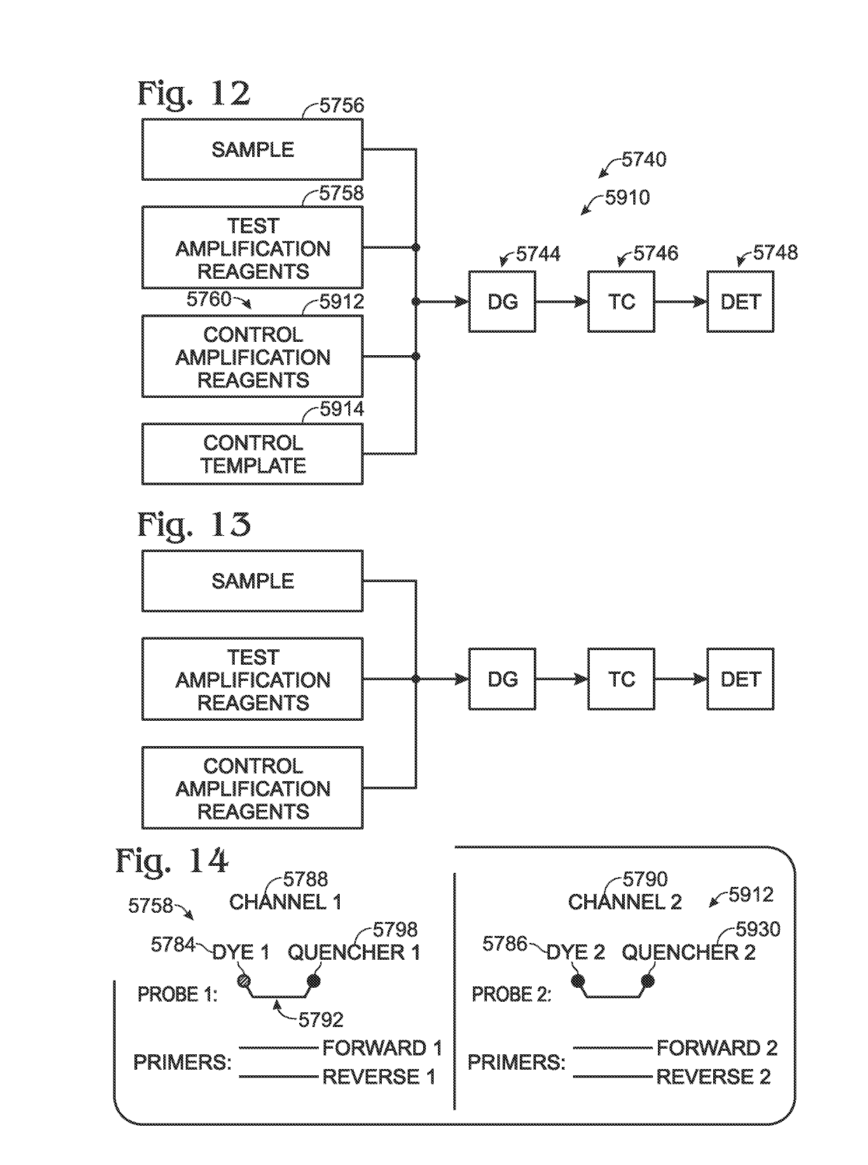

FIG. 13 shows selected aspects of system 5740 of FIG. 5, with the system in another exemplary configuration 5920 for testing amplification of at least a pair of nucleic acid targets in the same droplets. System configuration 5920 differs from configuration 5910 of FIG. 12 by including a different set of control amplification reagents 5922 (or a second set of test amplification reagents) and by the absence of an exogenous control template. Control reagents 5922 may amplify a control target that is known or expected to be present in sample 5756, and/or that has a known or expected representation with respect to a bulk nucleic acid population present in the sample (e.g., total DNA, total genomic DNA, genomic DNA from a particular species of organism, total RNA, total mRNA, etc.). In contrast, target reagents 5758 may amplify a test target that has an unknown presence in the sample and/or an unknown presence in with respect to the bulk nucleic acid population. In any event, amplification of the control target may be used to determine the quality of test data measured from a sample, such as to verify the quantitative accuracy of the test data and/or to determine the reliability of the test data. Furthermore, an amount of control target determined to be present in the sample may provide a standard against which an amount of test target determined to be present in the sample can be compared and/or normalized. In some embodiments, a control target is selected that is rare in the sample, such as a target representing a particular gene mutation. By selecting a rare control target, amplification of the control target can indicate the limit of detection of a test target and/or whether amplification of a low-abundance test target can occur. In some embodiments, the control target may be replaced by a second test target with an unknown presence in the sample (before testing).

FIG. 14 shows exemplary test target reagents 5758 and control target reagents 5912 (or 5922) that may be included in system configuration 5910 (or 5920) of FIG. 12 (or 13), to permit detection of amplification signals in a different detection channel (i.e., channels 1 and 2, respectively) for each nucleic acid target. Test target reagents for channel 1 are described above in relation to FIG. 7. Control target reagents 5912 (or 5922) may be similar in general structure to the test target reagents, but different with respect to the nucleic acid sequences of the primers and probes, to provide test target and control target specificity, respectively. Also, the test and control probes may include distinct dyes 5784, 5786 and/or distinct energy transfer partners 5798, 5930 (e.g., distinct quenchers suitable for the respective dyes). In other embodiments, at least one of the probes may be replaced by a reporter including an intercalating dye, such as SYBR® Green.

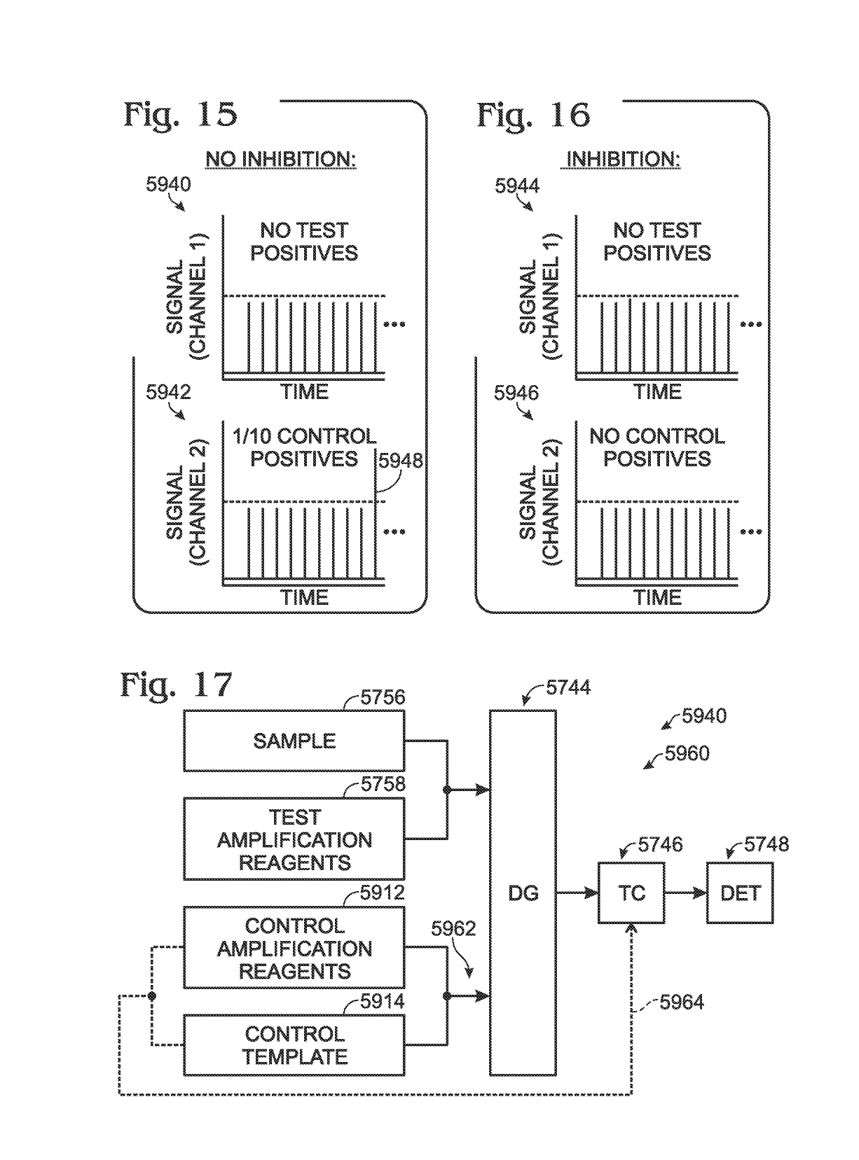

FIGS. 15 and 16 show representative portions of exemplary data that may be obtained using system configuration 5910 or 5920 and the reagents of FIG. 14. The figures show exemplary graphs 5940-5946 of fluorescence signals that may be detected over time from a flow stream of the system using different detection channels, namely, a test channel (channel 1) that detects test data and a control channel (channel 2) that detects control data. In FIG. 15, graph 5940 of the test data contains no positive droplet signals. In contrast, graph 5942 of the control data identifies positive droplet signals, such as a positive signal 5948, at a frequency of about one in ten. Thus, the control data demonstrates that amplification in the droplets is not inhibited substantially and suggests that the lack of positive signals from the test data is due to an absence or undetectable level of the test target in the sample. Accordingly, the control data supports and helps to validate the negative result in the test data. In contrast, control graph 5946 of FIG. 16 shows no amplification of the control target (a substantially larger data set may be analyzed to demonstrate that the control result holds). The control data of graph 5946 thus indicates that amplification of the test target also is inhibited (or the sample is defective, such as too dilute (configuration 5920)), and that the negative test result is not valid.

FIG. 17 shows selected aspects of system of FIG. 5, with the system in an exemplary configuration 5960 for testing amplification of a pair of nucleic acid targets in respective different (i.e., nonoverlapping) sets of droplets. Configuration 5960 may be similar to that of configuration 5910, except that control reagents 5912 and control template 5914 are not mixed with sample 5756 and test target reagents 5758. Instead, droplets containing the control reagents and the control template may be formed separately in the system, indicated at 5962, or may be supplied as pre-formed droplets that are introduced into the flow stream downstream of droplet generator 5744, indicated at 5964.

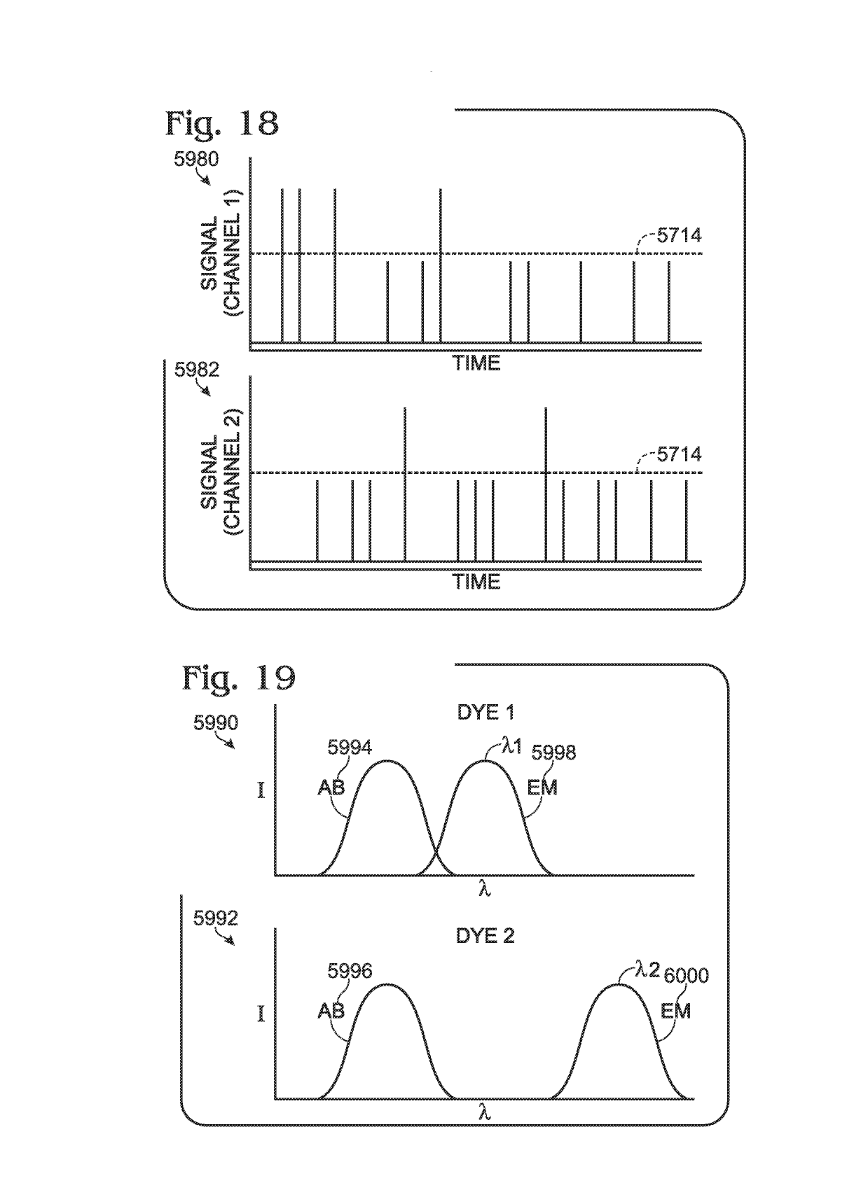

FIG. 18 shows a pair of exemplary graphs 5980, 5982 of fluorescence signals that may be detected over time from a flow stream of system configuration 5960 of FIG. 17 using different detection channels. Graph 5980 plots fluorescence signals detected from a first channel, which detects amplification, if any, of a test target. Graph 5982 plots fluorescence signals detected from a second channel, which detects amplification, if any, of a control target. Successful amplification of the control target, as shown here, may, for example, verify and/or measure aspects of the system, such as operation of the thermal cycler and/or the detection assembly, the quality of the reagents, fraction of amplification-positive droplets, or any combination thereof, among others.

In configuration 5960, the test and control reagents are disposed separately in distinct droplets, so droplet signals in the first and second channels are not coincident, that is, they are not detected at the same time. In other embodiments, the control target may, instead, be a second test target and the control template may, instead, be another sample (or the same sample). Thus, the use of at least two detection channels permits droplets for distinct amplification tests to be interspersed in the flow stream.

### V. EXEMPLARY MULTI-CHANNEL DETECTION

FIG. 19 shows a pair of graphs 5990, 5992 illustrating exemplary absorption and emission spectra of fluorescent dyes that may be used in the system of FIG. 5. The dyes are arbitrarily labeled dye 1 and dye 2, respectively. However, either dye may be used to detect test signals or control signals in the various system configurations disclosed herein. Moreover, while illustrated here for two distinguishable dyes, the system may be used for detection and analysis with three, four, or more distinguishable dyes.

Each graph plots the intensity of absorption (“AB”), indicated at 5994, 5996, and emission (“EM”), indicated at 5998, 6000, for the corresponding dye. The dyes may have substantially overlapping absorption spectra, such that the same wavelength of light may be utilized to excite both dyes. In contrast, the dyes may exhibit Stokes shifts (i.e., the difference (in wavelength or frequency units) between the maxima of the absorption and emission spectra) of different magnitudes. For example, dye 1 may exhibit a smaller Stokes shift and dye 2 a larger Stokes shift, or vice versa. Accordingly, the emission spectra of the dyes may be substantially shifted with respect to one another. As a result, emission from the two dyes may be detected at least substantially independently of one another in different detection channels, such as a detection channel that detects light of a first wavelength or wavelength range (e.g., λ1) and another detection channel that detects light of a second wavelength or wavelength range (e.g., λ2).

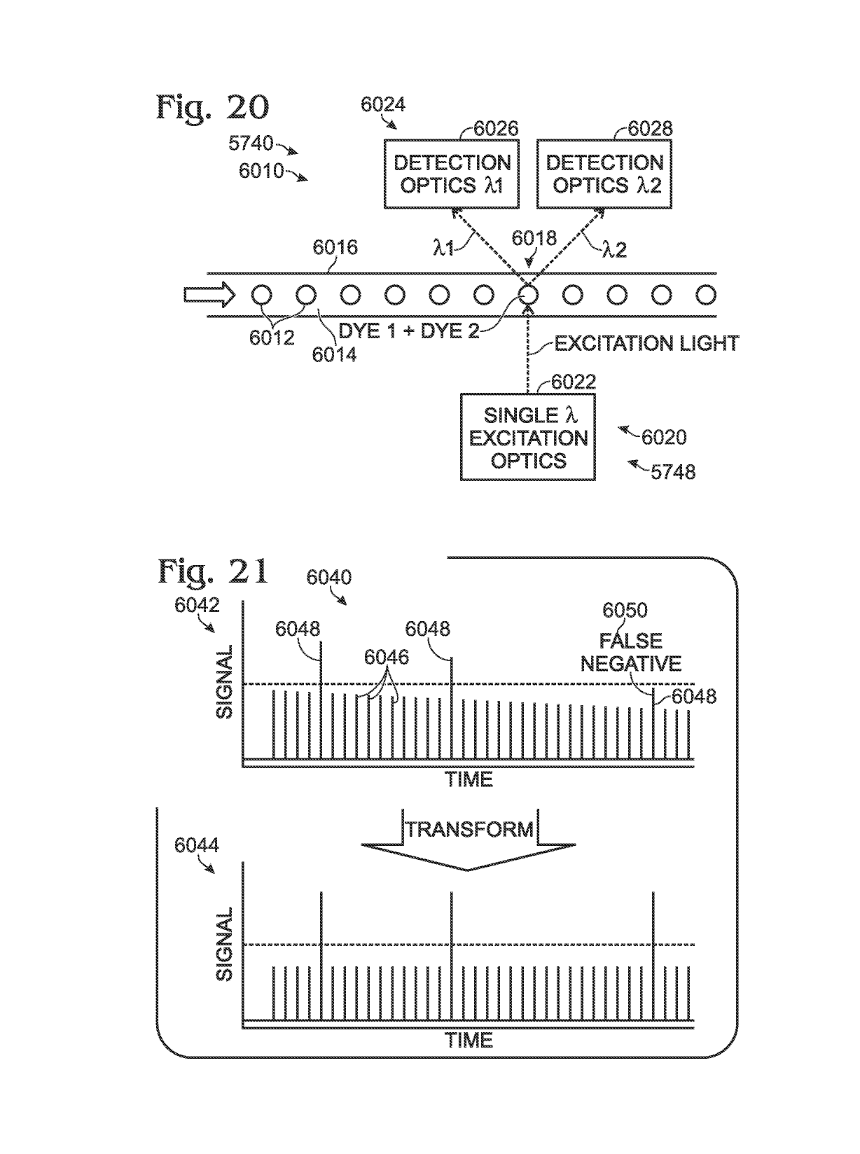

FIG. 20 is a schematic diagram illustrating exemplary use of the fluorescent dyes of FIG. 19 in an exemplary embodiment 6010 of system 5740 of FIG. 5. Droplets 6012 containing dyes 1 and 2, either in the same droplets or different sets of droplets, may be carried in a flow stream 6014 in a channel 6016. Flow stream 6014 may pass through a detection area 6018 established by an embodiment 6020 of detection assembly 5748.

Detection assembly 6020 may include a light source 6022 for exciting the fluorescent dyes in the droplets and at least one detector 6024 for detecting light emitted from the droplets. Light source 6022 may, for example, include an LED or laser that emits at least substantially a single wavelength of excitation light. Alternatively, or in addition, the light source may include at least one excitation optical filter that excludes other wavelengths of light emanating from the light source. Detector 6024 may be equipped with detection optics 6026, 6028 (e.g., beamsplitters, emission optical filters, separate detectors) that permit emitted light from the dyes to be detected separately.

Exemplary fluorescent dyes that may detected using system 6010 include a fluorescein derivative, such as carboxyfluorescein (FAM), and a PULSAR 650 dye (a derivative of Ru(bpy)₃). FAM has a relatively small Stokes shift, while PULSAR 650 dye has a very large Stokes shift. Both FAM and PULSAR 650 dye may be excited with light of approximately 460-480 nm. FAM emits light with a maximum of about 520 nm (and not substantially at 650 nm), while PULSAR 650 dye emits light with a maximum of about 650 nm (and not substantially at 520 nm). Carboxyfluorescein may be paired in a probe with, for example, BLACK HOLE Quencher™1 dye, and PULSAR 650 dye may be paired in a probe with, for example, BLACK HOLE Quencher™2 dye.

### VI. EXEMPLARY SELF-NORMALIZATION OF DROPLET SIGNALS

Test signals may be normalized using methods different from those described above in relation to FIGS. 8 and 11. In particular, the methods illustrated in FIGS. 8 and 11 involve transformation of test data with reference data detected (a) in a different detection channel (FIG. 8) or detected (b) in different droplets (FIG. 11). This section describes methods that transform test data using aspects of itself rather than another data set.

FIG. 21 shows a flowchart 6040 illustrating an exemplary method of correcting for system fluctuations during a test. The method involves processing a set of droplet test signals, shown in a first graph 6042, to produce a transformed set of test signals, shown in a second graph 6044. Negative test signals 6046 and positive test signals 6048 each should have respective constant values over time if there is no system variation. However, system variation, such as the negative drift over time illustrated in graph 6042, may produce false negatives, such as a false negative signal 6050, and/or false positives. Transformation of the test signals may be performed to correct for system variation before the test signals are used to estimate a presence of a test target in sample being tested. In particular, individual test signals may be transformed differently using the test data, accordingly to the temporal position of each test signal. For example, each test signal may be transformed using temporally proximate test data, such as normalization of each test signal with respect to a sliding window that averages a subset of the test signals including or adjacent the test signal. The subset of the test signals used may be provisionally negative, positive, or negative plus positive test signals, any of which may be re-assigned as negative/positive after transformation. For example, graph 6044 shows re-assignment of false negative signal 6050 as positive after transformation.

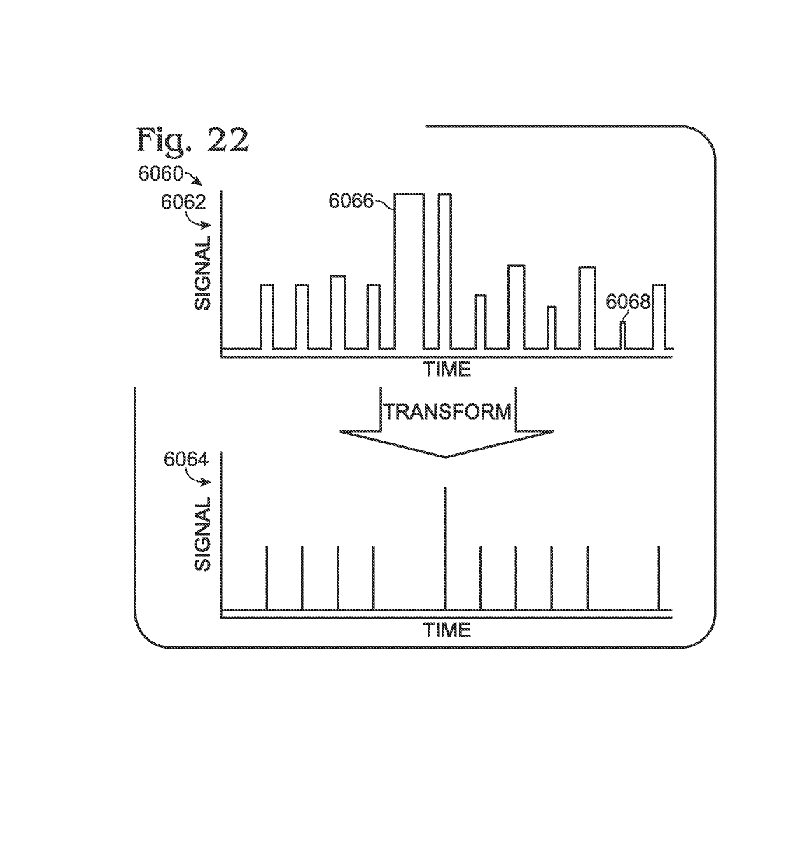

FIG. 22 shows a flowchart 6060 illustrating an exemplary method of transforming droplet signals based on the width of respective signal peaks providing the droplet signals. The flowchart involves graphs 6062, 6064, which represent test data before and after transformation, respectively.

Graph 6062 presents test data in which the width and height of each droplet peak is shown. (Here, each droplet peak is presented as a square wave to simplify the presentation. However, in other embodiments, each droplet peak may be detected as having any suitable shape, such as a wave with sloped leading and trailing sides.) The width of a droplet fluorescence peak may be used to determine the size and volume of each droplet, if droplet signals are detected in a flow stream with known flow rate, generally within a channel of fixed geometry. Knowing the volume of sample that is tested for amplification in droplets may be required for accurately determining the concentration/number of target molecules in the sample. If droplets of uniform size are desired, peak width may be used to identify droplets of sizes that are outside the desired range. For example, in FIG. 22, peaks 6066, 6068 having widths outside a predefined range are excluded from the data set. The droplet signals also may be transformed based on width, to provide transformed test data (i.e., graph 6064), that has been corrected for volume variation and/or variation in peak width.

### VII. EXAMPLES

The following examples describe selected aspects and embodiments of the present disclosure, including exemplary methods of gating droplet data and of determining the total number of droplets. These examples are intended for illustration and should not limit the entire scope of the present disclosure.

### Example 1

### Exemplary Identification of Accepted and Rejected Droplets

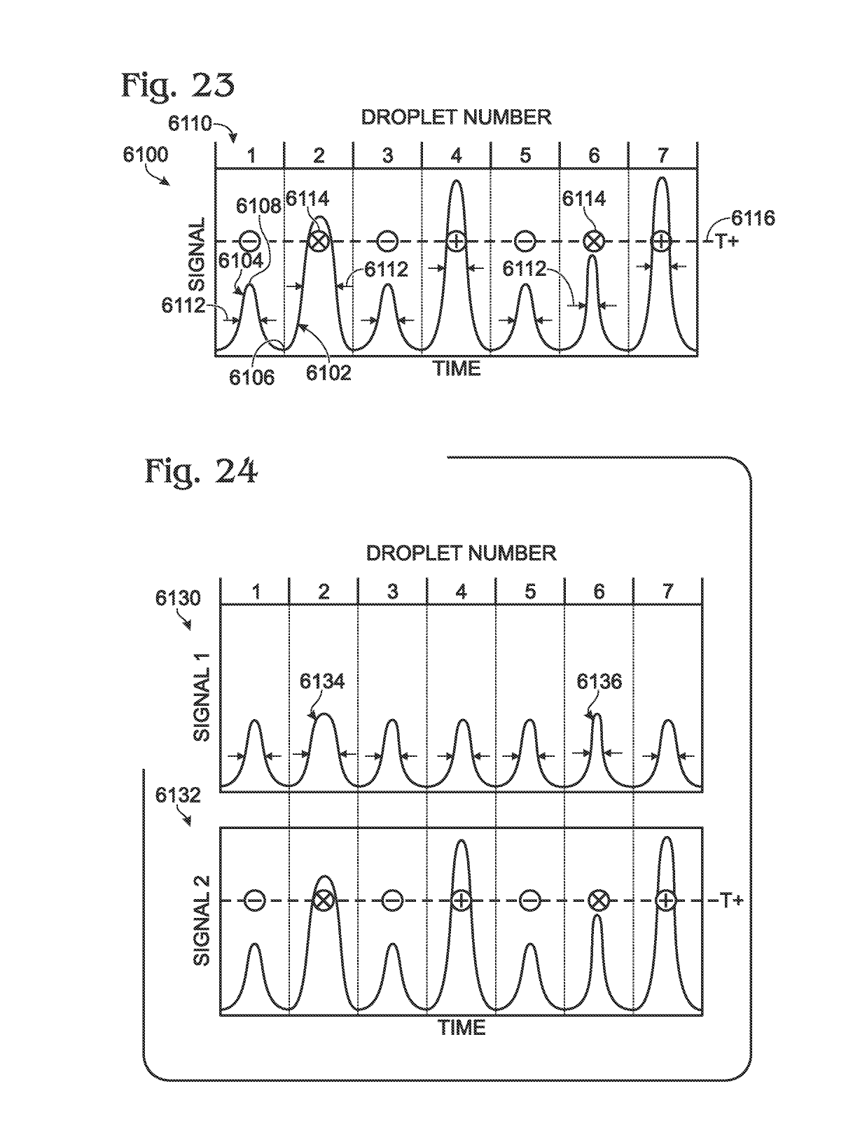

This example relates to culling data collected from droplets, as described above for FIG. 22; see FIGS. 23 and 24.

FIG. 23 shows an exemplary graph 6100 of a signal 6102 that may be measured with respect to time from a fluid stream containing droplets. In other embodiments, here and elsewhere in the present disclosure, the signal may be measured with respect to one or more spatial dimensions instead of time, such as when the signal is collected as an image of droplets. The signal may, for example, be generated by a fluorescence signal from an assay reporter (e.g., a probe) that reflects occurrence of a reaction, such as amplification of a nucleic acid target and/or other analyte.

Signal 6102 varies in strength over time as each droplet travels through a detection region where the signal is detected. In particular, the signal from each droplet (i.e., the “droplet signal”) generates a wave or peak 6104 as the intensity of signal 6102 increases above a baseline 6106 to a maximum or crest 6108 and then returns back to the baseline. Here, the graph shows a series of seven peaks, which are labeled at 6110 as droplet numbers 1 to 7.

Each droplet signal or peak has a height, which is a measure of how far the peak extends above baseline 6106, and a width, which, in this case, relates to the time interval during which the peak is detected. The peak width corresponds to the size of the droplet, generally its diameter. In other cases, such as when droplets are imaged, the peak width also may correspond to droplet diameter. In any event, the peak width may be defined in any suitable manner, such as a width 6112 of the peak at one-half of the peak height.

The droplet signals may be gated to exclude peaks (and thus corresponding droplets) that do not meet one or more predefined conditions. Each peak (or droplet signal) may be compared with the predefined condition to identify the corresponding droplet as an accepted droplet (an included droplet), if the peak (or droplet signal) meets the predefined condition, or a rejected droplet (an excluded droplet), if the peak (or droplet signal) does not meet the predefined condition. For example, the predefined condition may be a permitted size (such as a range of sizes) for a droplet. The size of a droplet is generally related to a width (e.g., signal duration) and/or area (e.g., total signal) of a peak formed by the signal for the droplet. Accordingly, the size may, for example, be defined by the width of each peak, such as width 6112.

FIG. 23 shows droplet numbers 2 and 6, which are marked by a circled “X” at 6114, having widths 6112 that fall outside the permitted width (such as a range of widths) and thus are identified as excluded/rejected droplets. The remaining droplets (droplets 1, 3-5, and 7) are identified as included/accepted droplets. This gating process may be utilized to improve the monodispersity of droplets analyzed, by excluding data from droplets that fall outside of a permitted size (e.g., a predefined size range). Generally, more accurate results are provided by using data from droplets that are closer to the same size.

The droplet signal from each accepted droplet may be used to determine whether each accepted droplet is positive or negative for the presence of a target. The height of each peak may be compared with a positive threshold (“T+”), indicated by a dashed line at 6116. If the peak extends above the threshold line, the peak (and the corresponding droplet) is deemed positive (indicated by a circled “+” on the threshold line), and if not, the peak (and the corresponding droplet) is deemed negative (indicated by a circled “−” on the threshold line). The concentration of an analyte and/or target in the accepted droplets may be determined by selectively using data from the accepted droplets, that is, without any contribution of data from the rejected droplets.

The differences in height of positive and negative peaks may affect the width measured from each, independent of droplet size. Accordingly, it may be desirable to use a droplet marker and an assay reporter that generate respective, distinguishable signals (e.g., signals measured from different wavebands of light). The droplet marker may be used to size droplets for exclusion of droplets. A signal detected from the droplet marker may have a strength that corresponds to a size of each droplet. Also, the strength of the signal detected from the droplet marker may be at least substantially independent of whether or not the target is present in each droplet. The assay reporter may be used to distinguish positive from negative droplets for a reaction (and/or analyte/target). The strength of a signal detected from the assay reporter may vary according to whether or not a target is present in each droplet.

FIG. 24 shows a pair of graphs 6130, 6132 of exemplary first and second signals (“signal 1” and “signal 2”) produced by a droplet marker and an assay reporter, respectively. The signals may be measured at respective distinct wavelength bands of light, which may or may not overlap. The droplet marker provides a peak size (such as height, width, and/or area, among others) for each droplet that corresponds to the droplet size. Accordingly, droplets having first-signal peaks that fail to meet a predefined condition (e.g., a permitted peak height, width, area, total signal, or the like) may be excluded from analysis of the corresponding second signal provided by the assay reporter. In other words, the first signal may be used to gate the second signal. Here, droplet numbers 2 and 6 generate first-signal peaks 6134, 6136 that fall outside a predefined peak width at half height. Accordingly, these droplets are identified as rejected droplets, as indicated by the circled “X” in graph 6132, and are not used to determine a concentration of a target. The remaining peaks produced by the second signal (graph 6132) may be used to identify positive and negative droplets and to obtain a concentration of a target, without any contribution of the second signal from the rejected droplets. For example, a fraction of the accepted droplets that are positive (or negative) may be used to calculate a concentration of the target in the accepted droplets, based on the target having a Poisson distribution among the accepted droplets.

### Example 2

### Exemplary Approaches to Determining Total Droplet Number

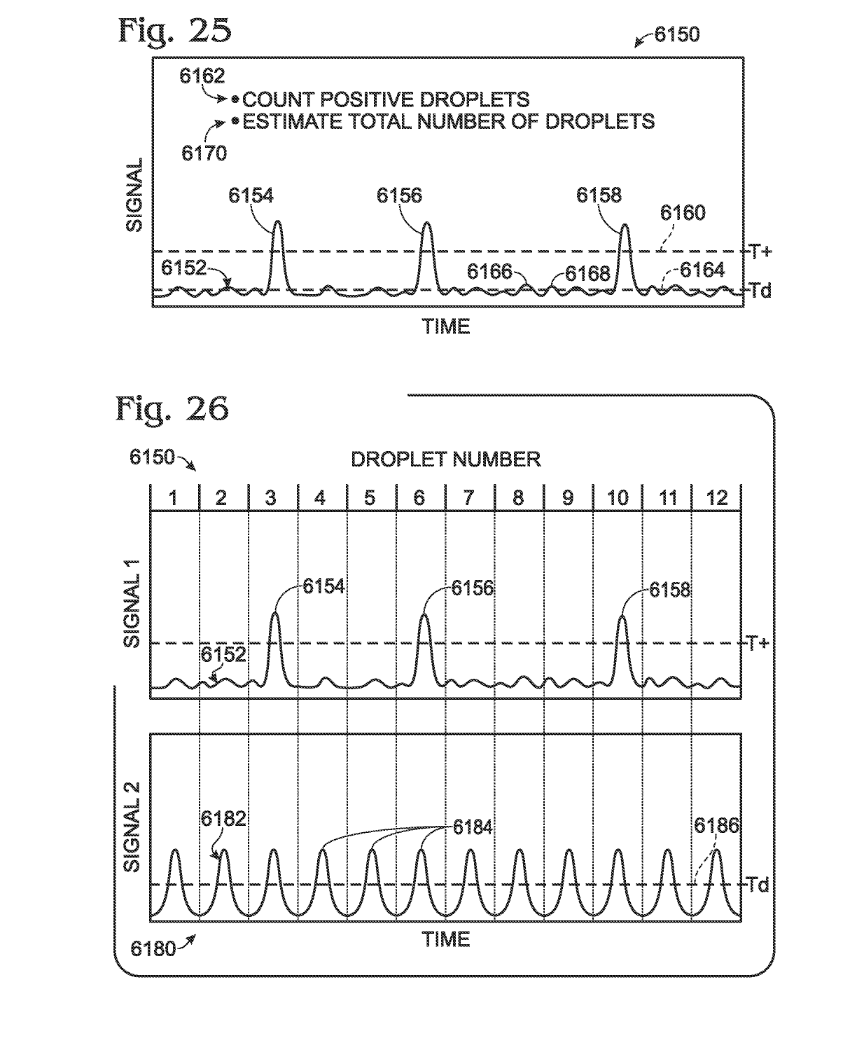

Signal peaks detected from negative droplets may be small and difficult to identify reliably. For example, an assay reporter with a low background (e.g., a fluorescent probe, such as a Taqman® probe) may produce a very weak signal (e.g., a strongly quenched signal) in the absence of reaction. Accordingly, counting the total number of peaks, and thus the total number of droplets analyzed, may not be practical with some assay reporters. This example describes exemplary approaches for determining the total number of droplets in an assay; see FIGS. 25-27.

FIG. 25 shows a graph 6150 of an exemplary signal 6152 that may be measured with respect to time from a fluid stream containing droplets. The signal may be produced by a reporter that is used to distinguish the presence or absence of at least one analyte or target molecule in individual droplets. A total of twelve equally spaced droplets produce signal 6152 presented in the graph. However, only three signal peaks 6154-6158 are identified reliably. Each peak exceeds a positive-droplet threshold 6160 (“T+”), and is identified as a positive droplet. The number of positive droplets may be counted, indicated at 6162.

In contrast, signal 6152 exhibits too much noise to provide reliable droplet identification with a droplet threshold 6164 (“Td”), because the droplet threshold must be set too close to the signal's baseline. Also, a bona fide peak 6166 detected from a droplet and a false peak 6168 produced by noise can be of comparable height.

The total number of droplets may be estimated, indicated at 6170, rather than counted. The estimate thus may be an external estimate. Estimation may be conducted by various strategies, generally without using the detected signal to perform the estimation and/or without counting all or any of the droplets. For example, the droplets may represent a known volume of an emulsion having an estimated or measured density of droplets in the known volume (i.e., the number of droplets per unit volume of the emulsion). Exemplary estimation of the density of droplets may be performed using, as a source of droplets, an emulsion containing packed droplets having a measured or assumed packing density. Alternatively, the droplets may be generated from a known total volume of aqueous phase and each may have a known average droplet volume, to permit the total number to be estimated by dividing the total volume by the average droplet volume. As another example, the droplets may be generated (and/or driven through a detection region) at a known rate for a known period of time, to permit the total number of droplets to be estimated by multiplying the rate by the period of time. In any event, the number of negative droplets may be determined by material balance: the counted number of positive droplets may be subtracted from the estimated total number of droplets to infer the number of negative droplets. For example, if it is estimated that there are 20,000 total droplets from which signal is detected, and 12,000 positive droplets are counted, then 8,000 of the droplets are inferred to be negative by material balance.

The concentration of an analyte or target in the droplets may be obtained from the counted number of positive droplets and the estimated number of total droplets. For example, a fraction of the total droplets that are positive (or negative) droplets may be calculated, and the fraction may be utilized to determine the analyte/target concentration based on the target/analyte having a Poisson distribution among the plurality of droplets.

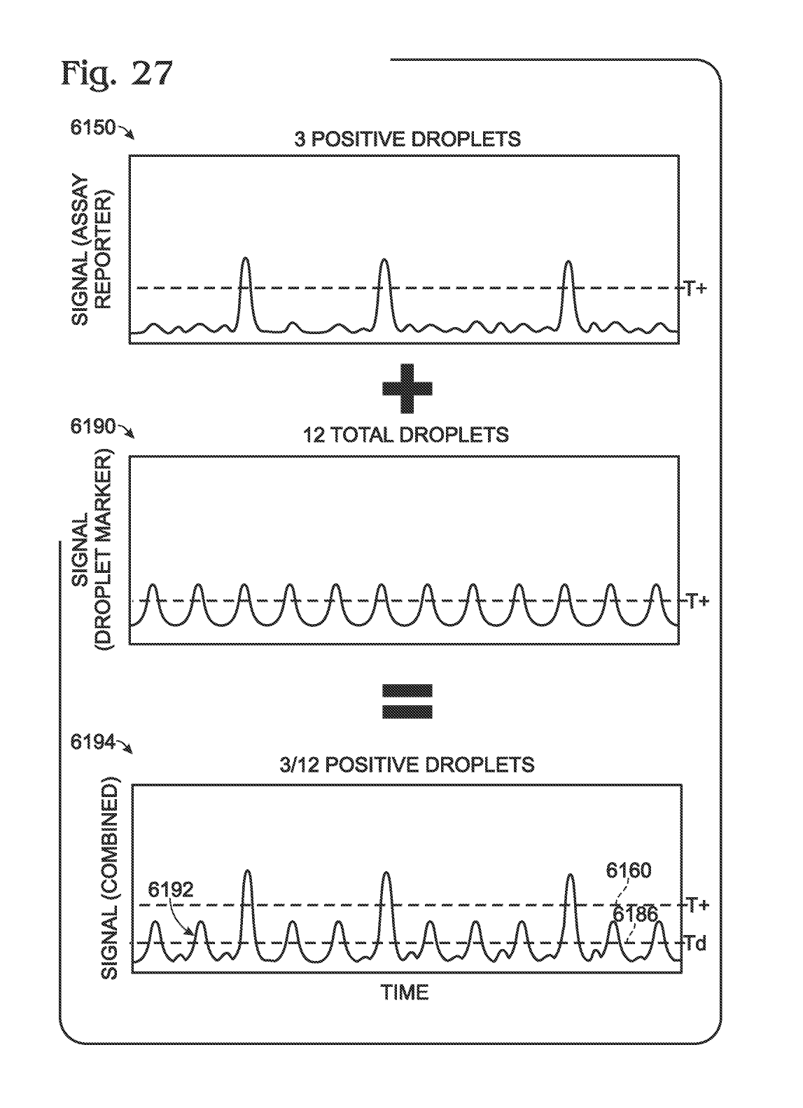

FIG. 26 shows data that permit counting the total number of droplets. The data are collected from a set of twelve droplets each carrying a pair of distinguishable dyes, which create respective first and second signals detected from distinct wavebands of light. Graph 6150 (also see FIG. 25) is formed with first signal 6152, and second graph 6180 is formed with a second signal 6182. Signals detected over the same time interval are presented by the graphs.

Graph 6150 permits signal peaks 6154-6158 to be identified as positive droplets, as described above for FIG. 25. However, the assay reporter used to create first signal 6152 does not permit reliable identification of peaks for the other nine droplets (i.e., droplet numbers 1, 2, 4, 5, 7-9, 11, and 12). Accordingly, the total number of droplets cannot be counted accurately.

Graph 6180 presents second signal 6182 detected from a droplet marker present in each droplet. The droplet marker produces a distinct peak 6184 for every droplet, with the peak extending significantly above the signal baseline. According, a droplet threshold 6186 (“Td”) may be set that accurately identifies at least substantially all bona fide peaks 6184 (and thus all of the droplets, whether positive or negative based on the first signal). The total number of droplets may be counted accurately using the second signal, and the number of positive droplets may be counted using the first signal. A fraction of the droplets that are positive (or a fraction that are negative) may be calculated and a concentration of the target obtained using Poisson statistics. In some cases, the total number of droplets may be determined by counting only a fraction (e.g., a contiguous fraction) of the droplets for only a portion of the total detection time, and then calculating the total droplet number by dividing the number counted by the fraction counted (e.g., as determined by dividing the counting time by the total detection time).

FIG. 27 shows data that permits counting the total number of droplets. However, in contrast, to the approach presented in FIG. 26, the same detected signal may be used for counting positive droplets and total (positive and negative) droplets. Here, the data is collected from a set of twelve droplets each carrying an assay reporter and a droplet marker. The assay reporter, if present without the marker, generates data presented in graph 6150 (also see FIG. 25). The droplet marker, if present without the reporter, generates data presented in a second graph 6190. Graph 6190 is similar to graph 6180 of FIG. 26, except that the signal from the droplet marker is not detected in a second channel. Accordingly, when the droplets carry both the assay reporter and the droplet marker, a combined signal 6192 is detected from the droplets as presented in a graph 6194 that corresponds to the sum of the signals from graphs 6150 and 6190.

The combined signal is composed of a first integral portion and a second integral portion. The first integral portion is produced by the assay reporter and has an intensity that varies according to whether or not a target is present in each droplet. The second integral portion is produced by the droplet marker and has an intensity that is at least substantially independent of whether or not the target is present in each droplet. The second integral portion may (or may not) be predominant over the first integral portion of the signal if the target is absent from a droplet. Alternatively, or in addition, the first integral portion of the signal may (or may not) be predominant over the second integral portion of the signal if the target is present in a droplet.

Combined signal 6192 permits identification and counting of each peak produced by the droplets (e.g., by comparing the peak height with droplet threshold 6186). The combined signal also permits identification and counting of each peak produced by a positive droplet (e.g., by comparing the peak height with positive-droplet threshold 6160).

The assay reporter and the droplet marker used to create combined signal 6192 both may include a fluorophore. The respective fluorophores may be excited by the same wavelength band of light and/or may emit light of overlapping (and/or the same) wavelength ranges. Accordingly, combined emission of light from the fluorophores may be detected from the same wavelength band. In some cases, the reporter and the marker may include the same fluorophore, with emission from the fluorophore of the reporter indicating whether a droplet is positive or negative and emission from the fluorophore of the marker being substantially independent of whether the droplet is positive or negative. In exemplary embodiments, the reporter is a probe that includes an oligonucleotide conjugated to a fluorophore, and the marker includes the fluorophore without the oligonucleotide (and/or without a quencher conjugated to the oligonucleotide).

### Example 3

### Selected Embodiments

This example describes selected embodiments of the present disclosure related to methods of using controls and calibrations for droplet-based assays, in accordance with aspects of the present disclosure, presented without limitation as a series of numbered paragraphs.

1. A method of performing a droplet-based assay, comprising: (A) detecting a first signal and a second signal from a plurality of droplets; (B) identifying accepted droplets of the plurality for which the first signal meets a predefined condition and rejected droplets of the plurality for which the first signal does not meet the predefined condition; and (C) determining a concentration of a target in the accepted droplets based on the second signal from the accepted droplets, and without any contribution of the second signal from the rejected droplets.

2. The method of paragraph 1, further comprising a step of amplifying the target in one or more of the plurality of droplets before the step of detecting.

3. The method of paragraph 2, wherein the step of amplifying the target includes a step of cycling the droplets thermally.

4. The method of any of paragraphs 1 to 3, wherein the step of detecting includes a step of detecting a first signal from droplets that are flowing.

5. The method of paragraph 4, wherein the step of detecting includes a step of detecting a first signal from droplets serially.

6. The method of any of paragraphs 1 to 3, wherein the step of detecting includes a step of detecting an image of droplets.

7. The method of any of paragraphs 1 to 6, wherein the predefined condition corresponds to a permitted size for the droplet, and wherein the permitted size corresponds to a permitted diameter for the droplets.

8. The method of any of paragraphs 1 to 7, wherein the first signal detected from each droplet forms a peak, and wherein the width of the peak is measured at about one-half of the peak height.

9. The method of any of paragraphs 1 to 8, wherein the first signal is a fluorescence signal.

10. A method of performing a droplet-based assay, comprising: (A) generating a plurality of droplets containing an assay reporter and a droplet marker; (B) detecting from the plurality of droplets a signal representing combined emission of light from the assay reporter and the droplet marker, wherein the assay reporter provides a first integral portion of the signal having an intensity that varies according to whether or not a target is present in a droplet, and wherein the droplet marker provides a second integral portion of the signal having an intensity that is at least substantially independent of whether or not the target is present in a droplet; (C) counting a number of the plurality of droplets that are positive or that are negative for the target based on the signal; (D) determining a total number for the plurality of droplets based on the signal; and (E) obtaining a concentration of the target based on the counted number of droplets and the total number of droplets.

11. The method of paragraph 10, wherein the assay reporter and the droplet marker each include a same fluorophore.

12. The method of paragraph 10 or 11, wherein the assay reporter includes an oligonucleotide and a fluorophore.

13. The method of any of paragraphs 10 to 12, further comprising a step of illuminating droplets with electromagnetic radiation capable of producing the combined emission of light from the assay reporter and the droplet marker.

14. The method of any of paragraphs 10 to 13, wherein the droplet marker is selectively localized near or at a perimeter of each droplet.

15. The method of paragraph 14, wherein the droplet marker is selectively localized in or adjacent a skin that encapsulates the droplets.

16. The method of any of paragraphs 10 to 13, wherein the droplet marker is distributed at least substantially uniformly throughout each droplet.

17. The method of any of paragraphs 10 to 16, wherein the step of detecting includes a step of detecting a signal from droplets that are moving.

18. The method of any of paragraphs 10 to 17, wherein the step of detecting includes a step of detecting the signal from droplets serially.

19. The method of any of paragraphs 10 to 17, wherein the step of detecting includes a step of detecting an image of droplets.

20. The method of any of paragraphs 10 to 19, wherein the target is a nucleic acid target.

21. The method of any of paragraphs 10 to 20, wherein the step of obtaining includes a step of determining a fraction of the plurality of droplets that are positive or a fraction that are negative for the target.

22. The method of any of paragraphs 10 to 21, wherein the step of obtaining a concentration includes a step of determining a concentration of the target based on the target having a Poisson distribution in the plurality of droplets.

23. The method of any of paragraphs 10 to 22, further comprising a step of amplifying the target in one or more of the plurality of droplets before the step of detecting.

24. The method of paragraph 23, wherein the step of amplifying the target includes a step of cycling the plurality of droplets thermally.

25. A method of performing a droplet-based assay, comprising: (A) detecting a signal from a plurality of droplets; (B) determining which of the droplets are positive for a target based on the signal; (C) counting the positive droplets to establish a number of positive droplets; (D) estimating a total number for the plurality of droplets; and (E) obtaining a concentration of the target based on the number of positive droplets and the total number of droplets.

26. The method of paragraph 25, wherein the signal is detected from droplets that are moving.

27. The method of paragraph 26, wherein the signal is detected serially from droplets flowing through a detection region.

28. The method of paragraph 25 or 26, wherein the step of detecting includes a step of detecting an image of droplets.

29. The method of any of paragraphs 25 to 28, wherein the signal is a fluorescence signal.

30. The method of any of paragraphs 25 to 29, wherein the target is a nucleic acid target.

31. The method of paragraph 30, further comprising a step of amplifying the nucleic acid target in one or more of the plurality of droplets before the step of detecting.

32. The method of paragraph 31, wherein the step of amplifying the nucleic acid target includes a step of cycling the droplets thermally.

33. A method of performing a droplet-based assay, comprising: (A) detecting a first signal from a plurality of droplets; (B) determining which of the droplets are positive for a target based on the first signal; (C) counting the positive droplets to establish a number of positive droplets; (D) detecting a second signal from the plurality of droplets, wherein the second signal has an intensity corresponding to a size of each droplet and substantially independent of whether or not the target is present in the droplet; (E) determining a total number for the plurality of droplets based on the second signal; and (F) obtaining a concentration of the target in the plurality of droplets based on the number of positive droplets and the total number of droplets.

34. The method of paragraph 33, wherein the first signal has an intensity that varies according to whether or not the target is present in a droplet.

35. The method of paragraph 33 or 34, wherein the step of detecting a first signal includes a step of detecting a first signal from droplets that are flowing.

36. The method of paragraph 35, wherein the step of detecting a first signal includes a step of detecting a first signal from droplets serially.

37. The method of any of paragraphs 33 to 35, wherein the step of detecting a first signal includes a step of detecting an image of droplets.

38. The method of any of paragraphs 33 to 37, wherein the first signal is a fluorescence signal.

39. The method of any of paragraphs 33 to 38, wherein the target is a nucleic acid target.

40. The method of paragraph 39, further comprising a step of amplifying the nucleic acid target in one or more of the plurality of droplets before the step of detecting.

41. The method of paragraph 40, wherein the step of amplifying the nucleic acid target includes a step of cycling the droplets thermally.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A method of performing a droplet-based assay, comprising:
detecting a first signal and a second signal from a plurality of droplets;
identifying accepted droplets of the plurality for which the first signal meets a predefined condition and rejected droplets of the plurality for which the first signal does not meet the predefined condition; and
determining a concentration of a target in the accepted droplets based on the second signal from the accepted droplets, and without any contribution of the second signal from the rejected droplets.

2. The method of claim 1, wherein the first signal has an intensity that corresponds to a size of each droplet and that is at least substantially independent of whether or not the target is present in the droplet.

3. The method of claim 1, wherein the predefined condition corresponds to a permitted size for the droplet.

4. The method of claim 1, wherein the first signal detected from each droplet forms a peak, and wherein the predefined condition is a permitted width of the peak.

5. The method of claim 1, wherein the step of determining includes a step of determining a fraction of the accepted droplets that are positive or a fraction that are negative for the target.

6. The method of claim 5, wherein the step of determining a fraction includes a step of counting droplets that are positive or that are negative for the target.

7. The method of claim 1, wherein the second signal has an intensity that varies according to whether or not the target is present in a droplet.

8. A method of performing a droplet-based assay, comprising:
generating a plurality of droplets containing an assay reporter and a droplet marker;
detecting from the plurality of droplets a signal representing combined emission of light from the assay reporter and the droplet marker, wherein the assay reporter provides a first integral portion of the signal having an intensity that varies according to whether or not a target is present in a droplet, and wherein the droplet marker provides a second integral portion of the signal having an intensity that is at least substantially independent of whether or not the target is present in a droplet;
counting a number of the plurality of droplets that are positive or that are negative for the target based on the signal;
determining a total number for the plurality of droplets based on the signal; and
obtaining a concentration of the target based on the counted number of droplets and the total number of droplets.

9. The method of claim 8, wherein the second integral portion of the signal for a droplet is greater than the first integral portion of the signal for the droplet if the target is absent from the droplet.

10. The method of claim 8, wherein the first integral portion of the signal for a droplet is greater than the second integral portion of the signal for the droplet if the target is present in the droplet.

11. The method of claim 8, wherein the assay reporter and the droplet marker each include a same fluorophore.

12. The method of claim 8, wherein the step of determining a total number includes a step of counting each droplet.

13. The method of claim 8, wherein the step of determining a total number includes a step of counting a portion of the plurality of droplets and a step of estimating the total number based on the portion counted.

14. The method of claim 8, wherein the light is visible light.

15. A method of performing a droplet-based assay, comprising:
detecting a signal from a plurality of droplets;
determining which of the droplets are positive for a target based on the signal;
counting the positive droplets to establish a number of positive droplets;
estimating a total number for the plurality of droplets; and
obtaining a concentration of the target based on the number of positive droplets and the total number of droplets.

16. The method of claim 15, wherein the step of estimating a total number is performed without use of the detected signal.

17. The method of claim 15, wherein the step of estimating a total number is performed without counting any droplets.

18. The method of claim 15, wherein the step of estimating a total number includes a step of counting a portion of the plurality of droplets before the step of detecting.

19. The method of claim 15, wherein the plurality of droplets are provided by a known volume of packed droplets, and wherein the step of estimating is based on the known volume and an estimated or measured packing density of droplets per unit volume in the packed droplets.

20. The method of claim 15, wherein the plurality of droplets are generated with a known total volume and have a known size, and wherein the step of estimating is based on the known total volume and the known size.

21. The method of claim 15, wherein the plurality of droplets are generated at a known rate for a known period of time, and wherein the step of estimating is based on the known generation rate and the known period of time.

22. The method of claim 15, wherein the step of obtaining includes a step of determining a fraction of droplets that are positive or a fraction that are negative for the target, and a step of finding a concentration of the target in the droplets based on the target having a Poisson distribution in the plurality of droplets.

23. A method of performing a droplet-based assay, comprising:
detecting a first signal from a plurality of droplets;
determining which of the droplets are positive for a target based on the first signal;
counting the positive droplets to establish a number of positive droplets;
detecting a second signal from the plurality of droplets, wherein the second signal has an intensity corresponding to a size of each droplet and at least substantially independent of whether or not the target is present in the droplet;
determining a total number for the plurality of droplets based on the second signal; and
obtaining a concentration of the target based on the number of positive droplets and the total number of droplets.

24. The method of claim 23, wherein the second signal is detected from a dye that is selectively localized near or at a perimeter of each droplet.

25. The method of claim 24, wherein the dye is selectively localized in, on, or about a skin that encapsulates droplets individually.

26. The method of claim 23, wherein the second signal is detected from a dye that is distributed at least substantially uniformly throughout each droplet.

27. The method of claim 23, wherein the step of determining a total number includes a step of counting each droplet of the plurality of droplets.

28. The method of claim 23, wherein the step of determining a total number includes a step of counting only a portion of the plurality of droplets and a step of estimating the total number based on the portion counted.

29. The method of claim 23, wherein the step of obtaining includes a step of determining a fraction of the droplets that are positive or a fraction that are negative for the target.

30. The method of claim 29, wherein the step of obtaining includes a step of finding a concentration of the target in the plurality of droplets based on a Poisson distribution of the target in the droplets.
